(19) United States
(12) Patent Application Publication
(10) Pub. No.: US 2025/0223638 A1
(43) Pub. Date: Jul. 10, 2025

(54) FLUORESCENCE DETECTION REAGENT, METHOD FOR PREPARING SAME, AND USE THEREOF

(71) Applicant: GeneMind Biosciences Co., Ltd., Shenzhen (CN)

(72) Inventors: Bin XIE, Shenzhen (CN); Meirong CHEN, Shenzhen (CN); Lei LIU, Shenzhen (CN); Weiyue CHEN, Shenzhen (CN); Wen WANG, Shenzhen (CN); Nannan ZHAO, Shenzhen (CN); Fang CHEN, Shenzhen (CN); Lei SUN, Shenzhen (CN)

(21) Appl. No.: 18/853,348

(22) PCT Filed: Mar. 28, 2023
(86) PCT No.: PCT/CN2023/084270

§ 371 Date: Oct. 1, 2024

(30) Foreign Application Priority Data
Apr. 2, 2022 (CN) 202210340645.4

(51) Int. Cl. C12Q 1/6869; C12Q 1/6806

(52) U.S. Cl. CPC C12Q 1/6869; C12Q 1/6806

(57) ABSTRACT

Disclosed are a fluorescence detection reagent, a method for incorporating a labeled nucleotide by using the reagent, and a kit or a mixture system. The fluorescence detection reagent comprises an enzymatic deoxygenation system and a plurality of reducing agents, and does not comprise DABCO. The composition is suitable for an application scenario in relation to fluorescence signal detection, in particular an application comprising single molecule fluorescence signal detection.

### TECHNICAL FIELD

The present disclosure relates to the technical field of fluorescence detection, in particular to a fluorescence detection reagent, a method for preparing the same, and use thereof.

### BACKGROUND

The subject matter discussed in this section should not be regarded as prior art merely as a result of its mention in this section. Similarly, technical problems mentioned in this section or associated with the subject matter provided as background should not be considered as having been previously recognized in the prior art. The subject matter in this section only represents different methods, which may themselves correspond to specific embodiments of technical solutions in the claims.

Detection of biomolecules with photoluminescent molecular markers based on optical systems is often used in scientific research and diagnostic applications. Take molecular diagnosis as an example. Fluorescent molecules are often used to modify nucleotides and/or oligonucleotides, etc., to realize detection and characterization of the structure of nucleic acids.

For example, in sequencing by synthesis (SBS) technology, a nucleotide molecule having an inhibiting group and a fluorescent label is excited by a light source of a specific wavelength and generates fluorescence after binding to an extended strand, and the signal is detected by an optical imaging system. By repetition of this step again and again, the biochemical signals are converted into electronic signals and the sequence determination of the nucleic acids is achieved based on the processing of these electronic signals.

However, during multiple repetitions of signal acquisition, usually the signal may weaken, and even the signal cannot be acquired at the original signal location. There are several possible causes for such cases. For example, the light signal intensity weakens due to the phasing in the amplification sequencing or a decrease in reaction efficiency (see WO2006/064199 and LakowiczJR, Principles of fluorescence spectroscopy, 3rd edition, Springer, 2006).

The influence of this phenomenon is particularly serious for SBS-based single-molecule sequencing, especially for platforms that realize single-molecule sequencing based on imaging of target nucleic acid molecules on the surface of the solid carrier. Generally, single-molecule sequencing does not involve the amplification of target nucleic acid molecules, i.e., the amplification of detection signals; in addition, a target nucleic acid molecule usually carries only one or a few fluorescent molecules, so it will be appreciated that, for single-molecule sequencing, the detection of each target nucleic acid molecule depends on the detection of the signal of an individual fluorescent molecule carried by the nucleic acid molecule by an imaging system, i.e., it is acknowledged that obtaining an image with a high signal-to-noise ratio is a difficulty in realizing single-molecule sequencing; in other words, during the process of acquiring or detecting the single-molecule fluorescence signal, the intensity and stability of the single-molecule fluorescence signal directly affect the accuracy and apparent read length of sequencing and directly affect the completion of the single-molecule sequencing. Here, a series of biochemical phenomena that may occur under laser irradiation need to be considered. The biochemical phenomena include damages to DNA molecules and effects on fluorescence brightness and fluorescence stability of labeled fluorescent molecules caused by laser irradiation. It will be appreciated that when the fluorophore is excited to enter an excited singlet state (S1), it may return to the ground state (S0) by releasing photons (i.e., fluorescence); it is also possible to change state by other means, e.g., by intersystem crossing (ISC), to enter an excited triplet state (T1), and it takes longer (lifetime) to return from the triplet state to the ground state, which leads to a decrease in brightness. Furthermore, the photo-induced damage or the like caused by light irradiation also causes a decrease in brightness and even makes it difficult to acquire the fluorescence signal. In addition, molecules in the S1 and T1 states have high reactivity and can react with free radicals and other molecules in the solution, resulting in photobleaching. Meanwhile, a nucleic acid may be broken/degraded under the condition of photoinduction, so that a DNA strand to be tested is damaged and thereby sequencing cannot be performed.

Thus, in the process of detecting a fluorescence molecule, it is often necessary to place the target nucleic acid molecule/fluorescent molecule in a specific solution environment to maintain the stability of the fluorescence signal, enhance the fluorescence signal, and/or reduce the damage of photochemical reactions to the surface of the solid carrier or substances including DNA strands, nucleotides (bases), enzymes, etc., on the surface of the solid carrier that participate in or catalyze the reactions.

The so-called solution environment is namely an environment that facilitates the imaging acquisition of fluorescence signals and is suitable for the detection of target nucleic acid molecules. In particular, a reagent or formulation for the detection of single molecules, including a method for preparing the reagent or formulation, needs to be provided or improved.

### SUMMARY

To solve at least one of the above-mentioned technical problems to at least some extent or to provide a practical commercial means, embodiments of the present disclosure provide a method for incorporating a labeled nucleotide, a fluorescence detection reagent and use thereof, and a method for preparing the fluorescence detection reagent.

For a platform that is based on the SBS principle and realizes sequencing by acquiring optical signals through imaging and processing and converting image signals into base/nucleotide information, one sequencing run usually includes multiple cycles of reactions/multiple repeated reactions, and each cycle of reaction includes introduction of optically detectable labels such as fluorescent labels, excitation of fluorescence signals, acquisition and imaging of signals and removal of fluorescent labels.

It will be appreciated that, at least because no biochemical reaction can proceed with 100% efficiency, after multiple cycles of reactions, the reaction conditions/environments are increasingly unfavorable for the subsequent reactions/detections, which is represented by the increasingly lower signal-to-noise ratio of the acquired images of the corresponding fields of view and the increasingly lower reliability of the correspondence between the signals on the images and the target nucleic acid molecules.

Although many factors affecting the above-described problems cannot be clarified and it is difficult to grasp the relationship between the factors, the inventors believe that the above-described problems can be significantly improved if a solution environment favorable for fluorescence radiation and fluorescence signal detection can be provided.

To this end, the inventors make the following summarization and provide the solution system based on the disclosure of the publications (U.S. Pat. Nos. 7,282,337 and 7,666,593) found and considered by themselves to be closest to the technology employed in the development platform, including the employed solution environment, description and test results in examples, in combination with their own understanding of the fluorescence irradiation principle and the factors reported to influence fluorescence irradiation and knowledge of the oxidation or reduction reaction or substance as well as their own findings in numerous comparative tests.

Specifically, the inventors believe that in the single-molecule sequencing process based on imaging of the surface of the solid substrate, the solution system (sometimes also referred to as “imaging solution”) in which the fluorescent molecule is placed should preferably be capable of increasing the intensity of the fluorescence signal, such as inhibiting the ISC process, reducing the quenching probability, avoiding or reducing the photo-induced damage, etc., so as to increase the fluorescence quantum efficiency of the fluorescent molecule and prolong the fluorescence lifetime and thereby maintain the intensity (brightness) and stability of the fluorescence signal in the detection.

Furthermore, preferably, the imaging solution should contain an oxygen scavenging system. This is because oxygen molecules can participate in photochemical processes and thereby result in the quenching of the fluorescent molecules. For example, oxygen molecules can react directly with excited fluorescent molecules and thereby result in the quenching of the fluorescent molecules, and free radicals can also be generated and result in the quenching of the fluorescent molecules (Lakowicz J R, Principles of fluorescence spectroscopy, Springer science & business media; 2013 Apr. 17; J. Vogelsang et al., A reducing and oxidizing system minimizes photobleaching and blinking of fluorescent dyes, AngewandteChemie International Edition, vol. 47, no. 29, pp. 5465-5469, 2008); meanwhile, oxygen molecules can have obvious effect on guanine in DNA (Helmut Sies et al., Singlet oxygen induced DNA damage, Mutation Research DNAging, 1992; H. Piwoński et al., Optimal oxygen concentration for the detection of single indocarbocyanine molecules in a polymeric matrix, Chemical Physics Letters, 2005, 405 (4-6): 352-356) and amino acid residues such as tryptophan, cysteine and histidine in proteins (Davies M J, Reactive species formed on proteins exposed to singlet oxygen, Photochemical & Photobiological ences Official Journal of the European Photochemistry Association & the European Society for Photobiology, 2004, 3 (1): 17-25) by photochemical reactions, thus leading to the damage of the DNA strands, bases, enzymes and other important raw materials in the sequencing and thereby leading to the wrong identification of bases.

Furthermore, the oxygen scavenging system in the imaging solution should be an effective one, and solutions that are expected to contain an effective oxygen scavenging system can reduce the effect of oxygen molecules on fluorescent molecules and biomacromolecules and also reduce the proportion of fluorescent molecules in triplet state (C. Steinhauer et al., Superresolution microscopy on the basis of engineered dark states, Journal of the American Chemical Society, vol. 130, pp. 16840-16841, December 2008; R. Zondervan et al., Photoblinking of rhodamine 6g in poly(vinyl alcohol): Radical dark state formed through the triplet, The Journal of Physical Chemistry A, vol. 107, no. 35, pp. 6770-6776, 2003; T. Basche et al., Direct spectroscopic observation of quantum jumps of a single molecule, Nature, vol. 373, pp. 132-134, January 1995; T. Ha et al., Photophysics of fluorescent probes for single-molecule biophysics and super-resolution imaging, Annual review of physical chemistry, vol. 63, pp. 595-617, 2012); this is mainly because a large number of fluorescent molecules entering triplet state decreases the fluorescence quantum efficiency and increases the quenching probability.

Further, the imaging solution should include a redox system, and the component included in the redox system includes the component of the oxygen scavenging system, crosses with the component of the oxygen scavenging system, or is independent of the oxygen scavenging system. Numerous publications, including textbooks, disclose various oxidizing agents and reducing agents, such as methyl viologen (MV), p-nitrobenzyl alcohol, Trolox quinone (TXQ), ascorbic acid (AA), propyl gallate (nPG), water-soluble vitamin E (Trolox), mercaptoethanol (BME), dithiothreitol (DTT), mercaptoethylamine (MEA), and the like, each of which is independently capable of effectively eliminating fluorescent molecules in triplet state or allowing fluorescent molecules to be restored from triplet state to the ground state to improved the stability of the fluorescent dye (Holzmeister P et al., Geminate recombination as a photoprotection mechanism for fluorescent dyes, Angew Chem Int Ed Engl, 2014, 53 (22): 5685-5688; Vogelsang J et al., A reducing and oxidizing system minimizes photobleaching and blinking of fluorescent dyes, AngewandteChemie, 2010, 47 (29): 5261-5261; Aitken et al., An oxygen scavenging system for improvement of dye stability in single-molecule fluorescence experiments, Biophysical Journal, 2008, 94 (5): 1826-1835).

In addition, solutions containing the oxygen scavenging system and/or the redox system are dependent on suitable buffer systems during preparation, storage and use, and the pH, buffering capacity, ionic strength of solution, solution polarity, etc., may have an effect on the functional components and the properties of the composition and may also directly affect the quantum efficiency of the fluorescent molecule.

In summary, based on the conventional knowledge in the field, literature reports, guesswork and experiments and tests, the inventors found and summarized the above information, including the determination of some factors and directions that are considered to need attention. However, in view of unknown correlation between reagents/components, the presence or absence of other influencing factors, the correlation between the influencing factors, etc., it is difficult to predict whether an effective and stable fluorescence detection solution satisfying the above expectations can be made.

For this purpose, first, the inventors set or prepared the same detection conditions, detection environment and detection object and tested the solution system, i.e., the formulation “134 μL of HEPES/NaCl, 24 μL of 100 mM water-soluble vitamin E Trolox (prepared with 2-(N-morpholine) ethanesulfonic acid, i.e., the MES buffer system, pH 6.1), 10 μL of triethylenediamine DABCO (prepared with MES, pH 6.1), 8 μL of 2 M glucose, 20 μL of NaI (50 mM, prepared with water) and 4 μL of glucose oxidase” containing 30% acetonitrile, to which the fluorescent molecule was placed at the time of imaging as disclosed in the above-mentioned publication U.S. Pat. No. 7,282,337 or U.S. Pat. No. 7,666,593 (hereinafter, this formulation is referred to as “basic formulation”); specifically, multiple fields of view of a batch of solid substrates with target nucleic acid molecules attached to the surfaces thereof obtained in the same processing manner were imaged multiple times using the same optical imaging system (with the same light intensity and exposure time), including optimizing the concentrations of redox system components Trolox and/or DABCO therein. The results are basically as disclosed in the publications, and the obtained sequencing results have low application value, such as short read length and high error rate; for example, after changing the fluorescent dye Atto647N on the reaction substrate nucleotide to Atto532, the formulation does not enhance the brightness of Att532, but decreases its brightness and stability.

Furthermore, in the experiment of optimizing concentrations of components of the basic formulation, the inventors also found that as the sequencing progressed, the attenuation of the fluorescence signal acquired at each time was obvious, and the inventors guessed that it is possibly due to the following: the solution is exposed to air, the O₂ in the air continuously reacts with the oxygen scavenging system in the solution to generate a substance which acidifies the solution, and the continuous generation of the substance leads to a decrease in the pH of the solution due to the insufficient buffering capacity of the buffer system, so that the intensity of the fluorescence signal is significantly affected. Thus, the inventors believe that the buffering capacity of the buffer solution and the substance or combination of substances that affect the pH are factors that must be considered in the formulation optimization.

Further, the inventors evaluated the chemical properties of the components NaI and DABCO in the basic formulation and their influence/effect on the test solution system. In the basic formulation, the main role of NaI should be as a catalyst to effect decomposition of H₂O₂, while DABCO is a commonly used quenching agent for singlet O in fluorescence detection. It has been found in the test that: (1) NaI is unstable and easily oxidized and is prone to form precipitates in the aliquoting and storing process after preparation; moreover, surprisingly, the inventors have found that its removal from the formulation does not affect the working utility of the formulation, in other words, the presence or absence of NaI does not appear to affect or significantly affect the performance of the function and effect of the basic formulation; (2) more surprisingly, the inventors have also found that the sequencing results obtained from fluorescence imaging using a basic formulation that does not contain DABCO at the designated concentration are substantially the same as the sequencing results using the complete basic formulation.

In addition, the formulation optimization is performed by using a basic formulation not containing DABCO at the designated concentration as a “new basic formulation”, that is, the above listed oxidizing agents or reducing agents or analogs/derivatives thereof were added to the new basic formulation independently or in combination based on functions, redox ability and requirements for reaction environment, and whether a solution system containing a specific substance or combination of substances is suitable for the mixed use of fluorescent dyes with different water solubility or electric property (capable of making the fluorescent dyes in the system have high signal-to-noise ratio, increasing the stability of fluorescent dyes, etc.), whether it can reduce degradation of nucleic acids, whether it can have higher stability, etc., are considered, so as to obtain a formulation capable of resulting in significantly better sequencing results than the basic formulation or the new basic formulation.

Based on the unexpected findings and adjustment and optimization in multiple experiments described above, the inventors have developed the desired fluorescence detection solution system based on the new basic formulation.

In a certain embodiment, the present disclosure provides a method for incorporating a labeled nucleotide, which includes: (a) providing a hybridization complex, where the hybridization complex is a hybrid of a primer and a template molecule, the primer being configured to hybridize to a 3′ end of the template molecule and the template molecule being a single-stranded nucleic acid molecule; (b) subjecting a polymerase, a nucleotide analog and the hybridization complex to conditions suitable for a polymerization reaction to obtain an extension product by binding the nucleotide analog to the hybridization complex, the nucleotide analog including a sugar unit, a base, a cleavable blocking group and a fluorescent label linked together; (c) replacing a solution system of (b) with a fluorescence detection reagent including an enzymatic oxygen scavenging system and a plurality of reducing agents and no triethylenediamine (DABCO); (d) irradiating at least a portion of the hybridization complex and acquiring at least a portion of a signal from the fluorescent label in the presence of the fluorescence detection reagent; and (e) replacing the fluorescence detection reagent with a cleavage reagent to cleave the cleavable blocking group and the fluorescent label on the extension product, the cleavage reagent being used for cleaving the cleavable blocking group and the fluorescent label of the nucleotide analog.

The method includes the step of replacing the solution system of the extension step with a specific solution system (herein referred to as a fluorescence detection reagent), and the method including this step enables the fluorescent label to be placed in a solution system that facilitates its stable and efficient luminescence without affecting the template molecule/hybridization complex, facilitating accurate detection of the signal from the fluorescent label and, therefore, facilitating accurate identification of the type of nucleotide incorporated into the template molecule based on the detected signal.

The method is suitable for a platform for realizing sequencing by synthesis based on fluorescence imaging detection, in particular a single-molecule sequencing platform based on SBS. Specifically, the solution system in which the fluorescent label is placed includes an enzymatic oxygen scavenging system and a plurality of reducing agents and no DABCO. The solution system can rapidly and effectively remove oxygen and quench fluorescent molecules in triplet state in a mode of electron transfer and the like, so the reaction of the fluorescent molecules in triplet state and oxygen molecules is avoided, which facilitates the restoration of the fluorescent molecules to a ground state, so that an effective and stable detection environment/imaging environment is well provided; in addition, the existence of the solution system can stabilize the fluorescence signal, reduce or inhibit the damage of nucleic acids and prolong the quenching time of fluorescence molecules in the fluorescence detection process, thereby being beneficial to increasing the sequencing read length and improving the sequencing quality.

In another embodiment, the present disclosure also provides a composition or a reagent formulation, also referred to as a fluorescence detection reagent. The composition or the reagent formulation includes an enzymatic oxygen scavenging system and a plurality of reducing agents and no DABCO. The composition is provided by the inventors based on the unexpected findings described above and is suitable for applications involving detection of fluorescence signals, particularly applications involving detection of single-molecule fluorescence signals.

In still another embodiment of the present disclosure, further provided is use of the composition or the reagent formulation in the detection of fluorescence, in particular in the detection of single-molecule fluorescence signals.

Additional aspects and advantages of the embodiments of the present disclosure will be partially set forth in the following description, and will partially become apparent from the following description or be appreciated by practice of the embodiments of the present disclosure.

### BRIEF DESCRIPTION OF THE DRAWINGS

In order to more clearly illustrate the technical solutions in the embodiments of the present application, the drawings required for use in the description of the embodiments or the prior art will be briefly described below. It is obvious that the drawings in the description below are only some embodiments of the present application, and other drawings can be derived from these drawings by those of ordinary skill in the art without making creative efforts.

Among the drawings:

FIG. 1 shows the fluorescence quenching time of each experimental group and the control group in Example 10;

FIG. 2 shows the sequencing read lengths of each experimental group and the control group in Example 10;

FIG. 3 shows the CallRatio of experimental group 1 and experimental group 2 in Example 12;

FIG. 4 shows the apparent read length of each experimental group in Example 14;

FIG. 5 shows the change in pH value and dissolved oxygen value DO over time after the opening of the bottle containing the fluorescence detection reagent of the control group (left panel) and the imaging quality after 45 h (right panel) in Example 16;

FIG. 6 shows the change in pH value and dissolved oxygen value DO over time after the opening of the bottle containing the fluorescence detection reagent of the experimental group 1 (left panel) and the imaging quality after 45 h (right panel) in Example 16;

FIG. 7 shows the change in pH value and dissolved oxygen value DO over time after the opening of the bottle containing the fluorescence detection reagent of the experimental group 2 (left panel) and the imaging quality after 45 h (right panel) in Example 16;

FIG. 8 shows the change in pH value and dissolved oxygen value DO over time after the opening of the bottle containing the fluorescence detection reagent of the experimental group 3 (left panel) and the imaging quality after 45 h (right panel) in Example 16;

FIG. 9 shows the quenching curves of the experimental groups to which ascorbic acid at different concentrations was added in Example 18;

FIG. 10 shows the quenching time of the experimental groups to which ascorbic acid at different concentrations was added in Example 18;

FIG. 11 shows the sequencing read length distribution of the experimental groups to which 0 mM and 20 mM ascorbic acid were respectively added in Example 18;

FIG. 12 shows the read length distribution of the experimental groups to which ascorbic acid at different concentrations was added in Example 18;

FIG. 13 shows the comparison of the imaging quality of the experimental group 1 (right panel) and the control group (left panel) in Example 22;

FIG. 14 shows the fluorescence intensity corresponding to the fluorescence detection reagents at different pH conditions in Example 28;

FIG. 15 shows the apparent sequencing read lengths corresponding to the fluorescence detection reagents at different pH conditions in Example 28;

FIG. 16 shows the quenching curves corresponding to fluorescence detection reagents to which BME at different concentrations was added in Example 30;

FIG. 17 shows the quenching time corresponding to fluorescence detection reagents to which BME at different concentrations was added in Example 30;

FIG. 18 shows the quenching curves corresponding to fluorescence detection reagents to which

DTT at different concentrations was added in Example 30;

FIG. 19 shows the quenching time corresponding to fluorescence detection reagents to which DTT at different concentrations was added in Example 30;

FIG. 20 shows the quenching time corresponding to fluorescence detection reagents to which AA and MV at different concentrations were added under the same ratio conditions in Example 30;

FIG. 21 shows the quenching time corresponding to fluorescence detection reagents to which AA and MV were added in different ratios in Example 30;

FIG. 22 shows the quenching time corresponding to fluorescence detection reagents to which AA and MV were added in different ratios in Example 30;

FIG. 23 shows the sequencing read length distribution corresponding to fluorescence detection reagents with or without AA and MV added in Example 30;

FIG. 24 shows the fluorescence brightness corresponding to fluorescence detection reagents with or without AA and MV added in Example 30;

FIG. 25 shows the mapping results corresponding to fluorescence detection reagents with or without AA and MV added in Example 30.

### DETAILED DESCRIPTION

The technical solutions in the embodiments of the present application will be described below clearly and comprehensively in conjunction with the drawings in the embodiments of the present application. It is evident that the described embodiments are part of the embodiments of the present application, but not all of them. Based on the embodiments of the present application, all other embodiments obtained by those of ordinary skill in the art without creative efforts fall within the scope of protection of the present application.

Unless otherwise defined, all technical and scientific terms used herein have the same meaning as commonly understood by those skilled in the art. As used herein, the term “and/or” includes any and all combinations of one or more of the associated listed items.

As used herein, unless otherwise indicated, the term “nucleotide” refers to four types of natural nucleotides (e.g., dATP, dCTP, dGTP and dTTP, or ATP, CTP, GTP and UTP) or derivatives thereof, and is sometimes directly referred to as the base included (A, T/U, C and G). The nucleotide or base represented by the expression will be known to those of ordinary skill in the art from the context.

As used herein, the term “sequencing” refers to sequence determination, and is used interchangeably with “nucleic acid sequencing” and “gene sequencing” to refer to the determination of base order in nucleic acid sequences, including sequencing by synthesis (SBS) and/or sequencing by ligation (SBL), including DNA sequencing and/or RNA sequencing, including long fragment sequencing and/or short fragment sequencing (the long fragment and short fragment are defined relatively; for example, nucleic acid molecules longer than 1 Kb, 2 Kb, 5 Kb or 10 Kb may be referred to as long fragments, and nucleic acid molecules shorter than 1 Kb or 800 bp may be referred to as short fragments), and including double-end sequencing, single-end sequencing, paired-end sequencing, and/or the like (the double-end sequencing or paired-end sequencing may refer to the reading of any two segments or portions of the same nucleic acid molecule that are not completely overlapping).

The sequencing includes the process of binding nucleotides (including nucleotide analogs) to a template and acquiring the corresponding reaction signals. Some sequencing platforms where the binding of nucleotides to the template and the acquisition of reaction signals are conducted asynchronously/in real-time generally involve multiple cycles of sequencing to determine the order of multiple nucleotides/bases on the template. A “cycle of sequencing”, also referred to as a “sequencing cycle”, may be defined as one base extension of the four types of nucleotides/bases, and in other words, as the determination process of the base type at any given position on the template.

For sequencing platforms that achieve sequencing based on polymerization or ligation reactions, one cycle of sequencing may include the process of binding four types of nucleotides to the template at a time and acquiring the corresponding reaction signals. For platforms that achieve sequencing based on polymerization reaction, a reaction system includes reaction substrate nucleotides, a polymerase, and a template; a predetermined sequence (a sequencing primer) is bound to the template, and on the basis of the base pairing principle and the rationale of polymerization reaction, the added reaction substrate (nucleotides) is controllably connected to the 3′ end of the sequencing primer under the catalysis of the polymerase to achieve the pairing with the base at a corresponding position of the template. Generally, one cycle of sequencing may include one or more base extensions (repeats). For example, four types of nucleotides are sequentially added to the reaction system to each perform base extension and corresponding acquisition of reaction signals, and one cycle of sequencing includes four base extensions and four signal acquisitions; for another example, four types of nucleotides are added into the reaction system in any combinations (such as in pairs or in one-three combination), the two combinations each perform base extension and corresponding acquisition of reaction signals, and one cycle of sequencing includes two base extensions and four signal acquisitions; for yet another example, four types of nucleotides are added simultaneously to the reaction system for base extension and acquisition of reaction signals, and one cycle of sequencing includes one base extension and four signal acquisitions.

Sequencing can be performed through a sequencing platform, which may be selected from, but is not limited to, the Hiseq/Miseq/Nextseq/Novaseq sequencing platform (Illumina), the Ion Torrent platform (Thermo Fisher/Life Technologies), the BGISEQ and MGISEQ/DNBSEQ platforms (BGI) and single-molecule sequencing platforms. The sequencing method may be selected from single-read sequencing and double-end sequencing. The acquired sequencing results/data (i.e., read fragments) are referred to as reads, and the length of a read is referred to as read length.

The single molecule detection includes single-molecule sequencing and is detection which does not involve amplifying a signal to be tested. For example, in a sequencing platform which does not involve amplifying a target nucleic acid molecule, the molecule to be tested exists in the physical form of a single molecule or a few molecules, which are shown, in the signal acquisition result, such as images, as weak, relatively unstable signals which are easily interfered/submerged. The “single molecule” refers to one or a few molecules, typically no more than 10, such as 1, 2, 3 or 5 molecules.

As used herein, the term “solid substrate” may be any solid support useful for immobilizing nucleic acid sequences, such as nylon membranes, glass slides, plastics, silicon wafers, magnetic beads, and the like, and may sometimes be referred to as a reactor, chip, or flow cell.

According to an embodiment of the present disclosure, provided is a method for incorporating a labeled nucleotide, which includes the following steps: (a) providing a hybridization complex, where the hybridization complex is a hybrid of a primer and a template molecule, the primer being configured to hybridize to a 3′ end of the template molecule and the template molecule being a single-stranded nucleic acid molecule; (b) subjecting a polymerase, a nucleotide analog and the hybridization complex to conditions suitable for a polymerization reaction to obtain an extension product by binding the nucleotide analog to the hybridization complex, the nucleotide analog including a sugar unit, a base, a cleavable blocking group and a fluorescent label linked together; (c) replacing a solution system of (b) with a fluorescence detection reagent including an enzymatic oxygen scavenging system and a plurality of reducing agents and no triethylenediamine; (d) irradiating at least a portion of the hybridization complex and acquiring at least a portion of a signal from the fluorescent label in the presence of the fluorescence detection reagent; and (e) replacing the fluorescence detection reagent with a cleavage reagent to cleave the cleavable blocking group and the fluorescent label on the extension product, the cleavage reagent being used for cleaving the cleavable blocking group and the fluorescent label of the nucleotide analog. Unless otherwise indicated, the labeled nucleotide and nucleotide analog herein are equivalent.

The nucleotides used in SBS sequencing typically include engineered nucleotides, which are also commonly referred to as “nucleotide analogs”. Nucleotide analogs used in SBS sequencing are also commonly referred to as terminators, and terminators used in current development stages generally are reversible terminators. In addition to the sequentially linked phosphate group, pentose and base that nucleotides generally include, a reversible terminator generally also includes a cleavable blocking group that is capable of reversibly preventing incorporation/binding of the subsequent reversible terminator or nucleotide to the next position of the template and a cleavable detectable label that enables a signal to be generated upon incorporation/binding of the reversible terminator to the current position of the template for detection.

Based on the attachment position of the blocking group, reversible terminators that have been reported can be divided into three classes: one is that the blocking group is located at the 3′-OH of the pentose, i.e., the pentose 3′-O-blocking group, which makes it impossible to form a phosphodiester bond, the blocking group being, for example, azide; one is that the blocking group is located on the phosphoric acid side, which makes it impossible to form a phosphodiester bond; and one is that the blocking group is located on the base side of the nucleotide and blocks the formation of a phosphodiester bond to realize polymerization blocking based on charge and/or steric hindrance in a solution environment, the reversible terminator being also commonly called a virtual terminator.

In a specific example, the nucleotide analog includes four types of nucleotide analogs, dATP, dUTP or dTTP, dCTP, and dGTP; two of the four types of nucleotide analogs carry a fluorescent label X, and other two of the four types of nucleotide analogs carry a fluorescent label Y; the fluorescent label X and the fluorescent label Y are two types of fluorescent labels having different emission wavelengths, and the polymerization reaction in (b) includes two of the four types of nucleotide analogs carrying different fluorescent labels.

In a specific example, the nucleotide analog includes four types of nucleotide analogs, dATP, dUTP or dTTP, dCTP and dGTP, the four types of nucleotide analogs carry fluorescent labels having four different emission wavelengths, and the polymerization reaction in (b) includes the four types of nucleotide analogs.

In a specific example, the blocking group and the fluorescent label on the nucleotide analog are located on the same side of the base. More specifically, in an example, the 3′ position of the sugar unit on the nucleotide analog is-OH, i.e., the 3′ position of the pentose is in a natural state. This nucleotide analog can block the nucleotide from being incorporated/bonded to the next position of the template by non-physical blocking means such as steric hindrance and/or charge effect of the molecule, and the specific structure can be found in, e.g., the disclosure of WO2019105421A1.

In a specific example, the blocking group and the fluorescent label on the nucleotide analog are located on different sides of the base. More specifically, the blocking group on the nucleotide analog is located at the 3′ position of the sugar unit, i.e., the pentose 3′-OH of the nucleotide analog is modified to 3′-O-blocking group, and the specific structure can be found in, e.g., the disclosure of U.S. Pat. No. 7,057,026B2.

In a specific example, the method for incorporating a labeled nucleotide described above further includes the following step: (f) performing (b)-(e) at least once, where the specific number of times can be determined as required. Usually, the steps (b)-(e), i.e., one base extension or one cycle of sequencing, are performed for no less than 20, 30, 50, 100, 150, etc., times to obtain a sequence with a certain length (reads), so as to meet the requirements of application detection for various purposes.

In a specific example, the hybridization complex is attached to a substrate surface, and (d) includes irradiating the substrate surface with light of a specific wavelength to excite fluorescence from the fluorescent label on the substrate surface and acquiring at least a portion of the fluorescence from the fluorescent label. It will be appreciated that the specific wavelength described above can be adjusted according to the excitation wavelengths of different fluorescent labels. For example, the wavelength range used for common imaging detection of fluorescence signals is 500 nm-700 nm, and the optional fluorescent dye or dye combination suitable for the excitation wavelength range may be selected from Cy3, Alexafluor 532, HEX, Atto 532, ROX, Alexafluorosis 630, Cy5, Atto647N, BODIPY650, Cy 5.5, IF700 and Alex680. More specifically, for example, for ATTO647N, the excitation wavelength is 646 nm and the emission wavelength is 664 nm; for ATTO532, the excitation wavelength is 532 nm and the emission wavelength is 552 nm; for CY5, the excitation wavelength is 651 nm and the emission wavelength is 670 nm; for IF700, the excitation wavelength is 690 nm and the emission wavelength is 713 nm; for ROX, the excitation wavelength is 578 nm and the emission wavelength is 604 nm; for Alexa Fluor532, the excitation wavelength is 534 nm and the emission wavelength is 553 nm.

In a specific example, the fluorescent label includes at least one in a combination of ROX, ATTO532 and Alexa fluor532 and at least one in a combination of CY5, IF700 and ATTO647N. That is, one base extension or one cycle of sequencing involves the acquisition of signals from two or more types of fluorescent labels.

In a specific example, the fluorescent label is selected from a combination of fluorescent dyes with excitation wavelengths of about 550 nm and about 660 nm. For example, the fluorescent labels include ATTO532 and ATTO647N.

In a specific example, the enzymatic oxygen scavenging system is selected from a combination I including glucose and glucose oxidase, a combination II including glucose, glucose oxidase and catalase or a combination III including protocatechuic acid and protocatechuate 3,4-dioxygenase. The detection solution containing any enzymatic oxygen scavenging system can effectively remove oxygen in the solution and can reduce the influence of singlet oxygen on the luminescence of the fluorescent dye.

In a specific example, the reducing agent is selected from at least two of ascorbic acid, gallic acid, an analog or derivative of gallic acid, cyanuric acid and water-soluble vitamin E or a derivative of water-soluble vitamin E. The analog or derivative of gallic acid is, e.g., lower alkyl ester of gallic acid, such as methyl gallate, ethyl gallate, propyl gallate or a combination thereof. Thus, by using a combination of a plurality of reducing agents, it is possible to act on fluorescent dyes having different characteristics. At the same time, other reducing agents in the reagent components may be protected and/or the stability of the nucleic acid molecule may be enhanced.

In certain examples, during detection of the fluorescence signal, a signal from the fluorescent label is acquired by a camera to obtain an image; it will be appreciated that at least a portion of the signal from the fluorescent label appears as a spot on the image.

The “spot” on an image, also referred to as “dot” or “peak”, refers to a position on an image where the signal is relatively strong, e.g., where the signal is stronger than the surrounding signals, appearing as a relatively bright speckle or dot on the image. A spot occupies one or more pixels. The signal corresponding to a spot may come from the target molecule or from a non-target substance. Detection of “spots” includes detection of the optical signal from a target molecule, such as an extended base or base cluster.

In a specific example, the fluorescence detection reagent includes the combination I, ascorbic acid and gallic acid or the analog or derivative of gallic acid. The combination of ascorbic acid and gallic acid can improve the detected fluorescence signal intensity and imaging quality score (image score), allow the imaging quality to be more stable in the signal acquisition process, and reduce the decreasing amplitude of the imaging quality in the signal acquisition process, so that a better detection result can be obtained. The inventor guesses that the fluorescence detection reagent of this embodiment uses glucose and glucose oxidase as an oxygen scavenging system and has a high oxygen removal speed; it also contains ascorbic acid as a reducing agent, fluorescent dye in triplet state is quenched by an electron transfer method and thus its reaction with oxygen molecules is avoided. This is beneficial to the restoration of the fluorescent dye to a ground state, so that an effective and stable detection solution system is well formed, and during fluorescence detection, the fluorescence signal can be stabilized, nucleic acid damage is reduced or inhibited, and the quenching time of the fluorescent molecule is prolonged, thus increasing the sequencing read length and improving the sequencing quality.

The imaging quality refers to the initial analysis obtained by observing an image and counting the brightness, the score value and the like through a mini program after introduction of a fluorescence detection reagent. The evaluation method of the imaging quality (image quality) is not limited in this embodiment. Expectations may be established based on prior data or theories, with relatively high scores being assigned for meeting or approaching expectations and relatively low scores being assigned for not meeting or being far from expectations. For example, the identification or detection of spots on the image and the evaluation of the image quality can be performed, for example, by referring to the method disclosed in CN112285070A.

In a specific example, the fluorescence detection reagent includes 50 mM-300 mM glucose, 2 U/mL-20 U/mL glucose oxidase, 1 mM-200 mM ascorbic acid and 1 mM-20 mM gallic acid or the analog or derivative of gallic acid.

Preferably, in a specific example, the fluorescence detection reagent includes 80 mM-150 mM glucose, 8 U/mL-12 U/mL glucose oxidase, 1 mM-50 mM ascorbic acid and 1 mM-10 mM gallic acid or the analog or derivative of gallic acid.

In an example, the fluorescence detection reagent further includes water-soluble vitamin E (Trolox) or a derivative thereof, or a combination of water-soluble vitamin E or a derivative thereof and a quinone derivative. It is found in tests that the water-soluble vitamin E or the derivative thereof has a protection effect on fluorophore, but the freshly prepared fluorescence detection reagent containing water-soluble vitamin E or the derivative thereof has poor imaging performance on fluorescent dyes such as Atto532, and the fluorescence detection reagent that has been stored does not have the problem. The inventors guess that this may be because that the protection of the fluorophore by the water-soluble vitamin E or the derivative thereof requires the combined action of Trolox in reducing state and TX-quinone (TQ) in oxidized state. Therefore, it is speculated that most of Trolox in the freshly prepared fluorescence detection reagent is in a reducing state and TQ is not present, and thus a good condition for excitation of the fluorescent dye cannot be created.

Based on the above test findings and speculation, the inventors adjust the method for preparing a solution containing Trolox or a derivative thereof. For example, the ultraviolet irradiation is increased or the standing time in certain amount of air is increased; for another example, the combination of Trolox or the derivative thereof and a quinone compound is adopted and the ratio of Trolox or the derivative there and the quinone compound is adjusted, thus enhancing the brightness of dye in the detection solution and the control of the stability of the detection solution system, and facilitating the realization of the function/action of the detection solution.

In a specific example, a quinone derivative such as p-benzoquinone is added in a certain proportion to a freshly prepared fluorescence detection reagent containing Trolox or a derivative thereof; the p-benzoquinone is used for simulating Trolox in an oxidized state, so that the purpose of enhancing the signal intensity of fluorophore can be achieved and thereby the imaging quality can be improved, the sequencing read length can be increased, and the error rate can be reduced.

Preferably, in an example, the fluorescence detection reagent further includes water-soluble vitamin E and p-benzoquinone.

In a specific example, the water-soluble vitamin E has a concentration of 6 mM-12 mM and the p-benzoquinone has a concentration of 0.36 mM-0.96 mM.

In a specific example, the fluorescence detection reagent further includes cyanuric acid. Thus, images with better quality can be obtained. The inventors guess that cyanuric acid is present in the solution in a free form and can react with oxygen radicals preferentially over molecules to be tested, such as nucleic acids on the surface of a chip, so that the oxygen radicals generated after laser irradiation can be consumed, and thereby damage to nucleic acid chains is reduced or avoided and wrong identification of bases can be reduced.

In a specific example, the mass percent of the cyanuric acid is 0.0001%-0.001%.

Preferably, in an example, the mass percent of the cyanuric acid is 0.0003%-0.0009%.

In a specific example, the fluorescence detection reagent further includes at least one of adenosine monophosphate, cytidine monophosphate, guanosine monophosphate, uridine monophosphate and thymidine monophosphate. The inventors guess that similar to cyanuric acid, these components, independently or in combination, are present in the solution in a free form and can react with oxygen radicals preferentially over molecules to be tested, so that the oxygen radicals generated after laser irradiation can be consumed and thereby damage to nucleic acid chains is reduced or avoided and the error rate of sequencing is reduced.

In a specific example, the fluorescence detection reagent further includes 1 μM-50 μM 5′-adenosine monophosphate and 1 μM-50 μM guanosine-5′-monophosphate. Thus, images with better quality can be obtained, and sequencing results with better quality can be obtained based on the images.

In a specific example, the reagent components of the fluorescence detection reagent include a Tris (tris(hydroxymethyl)aminomethane) buffer system or a HEPES buffer system, and the fluorescence detection reagent has a pH of 6.5-8.5. By detecting the physicochemical properties of the fluorescence detection reagent at different times, it is found that its pH value is gradually reduced, and the dissolved oxygen value is gradually increased. Based on the fact that glucose oxidase and glucose are used as an oxygen scavenging system in the fluorescence detection reagent, it is determined that the reason for the pH reduction is that glucose is oxidized into gluconic acid. In the later stage of sequencing, glucose is depleted and the dissolved oxygen is increased. The pH can be maintained ≥7 for a long time by 4-(2-hydroxyethyl) piperazine-1-ethanesulfonic acid (HEPES) at a certain concentration, so that the stability of the fluorescence detection reagent is improved. In addition, it is further found that the use of tris(hydroxymethyl)aminomethane (Tris) instead of HEPES is more beneficial to the improvement of the stability of the fluorescence detection reagent.

In a specific example, the fluorescence detection reagent further includes acetonitrile (ACN), and the volume percent of acetonitrile is 5%-40%, preferably 18%-35%. It is found in tests that part of the reducing substances tested have poor solubility in an acetonitrile-free solution system; for example, the dissolution of water-soluble vitamin E or some quinone derivatives in an acetonitrile-free solution is difficult. By adding ≥10% (v/v) acetonitrile, the dissolution of the functional substances in the solution can be enhanced.

It is found in tests that the combined use of Tris and ACN enables the acquired images to be better in fluorescence signal intensity, imaging quality and error rate of detection results.

Specifically, in an example, Trolox is dissolved in acetonitrile, and then the solution is left to stand in air for more than 10 h before the other components are added, thus preparing a fluorescence detection solution I without p-benzoquinone. In another example, a solution containing Trolox at the same concentration, 0.36 mM-0.96 mM, e.g., 0.52 mM, p-benzoquinone, and other components at the same concentration as I is prepared and is not left to stand in air, thus obtaining a fluorescence detection solution II. The fluorescence detection solutions I and II are both used for sequencing, and the results show that both the two formulations can obtain good sequencing results and have equivalent effects.

In some tests, such as sequencing in which the reaction system includes multiple fluorescent dyes, it is found that the formulations in some of the above embodiments can enhance the signals of some of the fluorescent dyes while reducing the signals of other fluorescent dyes. However, from the sequencing data of each example, SBS sequencing involving multiple fluorescent dyes can be effectively achieved, and relatively speaking, read length with application value can be basically obtained and the error rate is not high.

In a specific example, the fluorescence detection reagent includes the following components: 50 mM-200 mM glucose, 2 U/mL-20 U/mL glucose oxidase, 1 mM-200 mM ascorbic acid, 1 mM-10 mM gallic acid, 6 mM-12 mM water-soluble vitamin E, 0.1 mM-1.0 mM p-benzoquinone, 0.0001 wt % to 0.001 wt % cyanuric acid, 1 μM-20 μM 5′-adenosine monophosphate, 1 μM-20 μM guanosine-5′-monophosphate, 100 mM-300 mM tris(hydroxymethyl)aminomethane and 10 v/v %-40 v/v % acetonitrile.

Preferably, in an example, the fluorescence detection reagent includes the following components: 80 mM-150 mM glucose, 8 U/mL-12 U/mL glucose oxidase, 1 mM-50 mM ascorbic acid, 1 mM-10 mM gallic acid, 6 mM-12 mM water-soluble vitamin E, 0.36 mM-0.96 mM p-benzoquinone, 0.0003 wt %-0.0009 wt % cyanuric acid, 4 μM-12 μM 5′-adenosine monophosphate, 4 μM-12 μM guanosine-5′-monophosphate, 100 mM-300 mM tris(hydroxymethyl)aminomethane and 18 v/v %-35 v/v % acetonitrile.

Preferably, allowing the volume percent of acetonitrile therein to be 28%-35% can enable the acquisition of a high-quality detection result.

In some preferred examples, the fluorescence detection reagent includes the following components: 80 mM-150 mM glucose, 8 U/mL-12 U/mL glucose oxidase, 1 mM-50 mM ascorbic acid, 1 mM-15 mM gallic acid or ethyl gallate or propyl gallate, 6 mM-15 mM water-soluble vitamin E, 0.36 mM-0.96 mM p-benzoquinone, 0.0003 wt %-0.0009 wt % cyanuric acid, 4 μM-12 μM 5′-adenosine monophosphate, 4 μM-12 μM guanosine-5′-monophosphate, 100 mM-300 mM tris(hydroxymethyl)aminomethane and 18 v/v %-35 v/v % acetonitrile.

Or, the fluorescence detection reagent includes the following components: 80 mM-150 mM glucose, 8 U/mL-12 U/mL glucose oxidase, 10 mM-100 mM ascorbic acid, 10 mM-15 mM ethyl gallate, 6 mM-12 mM water-soluble vitamin E, 0.0003 wt %-0.0009 wt % cyanuric acid, 4 μM-12 μM 5′-adenosine monophosphate, 4 μM-12 μM guanosine-5′-monophosphate, 100 mM-300 mM tris(hydroxymethyl)aminomethane and 18 v/v %-35 v/v % acetonitrile.

Or, the fluorescence detection reagent includes the following components: 80 mM-150 mM glucose, 8 U/mL-12 U/mL glucose oxidase, 50 mM-150 mM ascorbic acid, 10 mM-15 mM propyl gallate, 3-7 mM hydroquinone, 6 mM-12 mM water-soluble vitamin E, 0.0003 wt %-0.0009 wt % cyanuric acid, 4 μM-12 μM 5′-adenosine monophosphate, 4 μM-12 μM guanosine-5′-monophosphate, 100 mM-300 mM tris(hydroxymethyl)aminomethane and 18 v/v %-35 v/v % acetonitrile.

The formulations are each applied to nucleic acid sequencing, particularly sequencing involving detection of multiple fluorescent dyes, and it is found that the formulations can all result in good sequencing results and the quality of the sequencing results obtained by using the formulations is equivalent.

It will be appreciated that in the sequencing process of sequencing by synthesis, the reaction usually lasts for several hours, e.g., 12 h or more, the solution reagents are each mixed in advance and then used for sequencing, and as the sequencing progresses, the performance of the mixed reagents exposed to air gradually declines, so that it is difficult to meet the sequencing requirements. Moreover, as a premixed reagent kit, its components contain a large number of reducing substances. The reducing substances are gradually oxidized in the storage process, and finally, the reducing components in the components are completely or partially oxidized, so that it is difficult for the premixed reagent kit to still play its role when used for detection after storage for a certain period of time.

In some examples, the fluorescence detection reagent further includes liquid paraffin and/or silicone oil. The stability of the fluorescence detection reagent can be ensured for a longer time by sealing and storing with liquid paraffin or silicone oil. Moreover, the reagent transportation is facilitated.

In a specific example, an inert substance such as silicone oil or liquid paraffin is added in an amount of, e.g., 8-20 v/v %. Given that the density of any of the two substances is less than that of the aqueous solution, the substance(s) can separate air from the solution system, thus avoiding or reducing the entry of oxygen in the air into the solution to consume the oxygen scavenging reagent therein, cause changes in the reducing substances and/or undergo undesired reactions with substances or intermediates in the solution and thereby alter the pH of the solution and/or affect the performance of the solution system, etc. Therefore, these inert substances can increase the stability of the detection solution in use and prolong the shelf life of the detection solution.

It is found in tests that in the case that 10 v/v % silicone oil is added within 12 h after the preparation of reagents and the reagents are exposed in the air for 48 h before sequencing, the three preferred fluorescence detection reagent formulations of the above examples can still maintain performance stable and result in good sequencing results. Moreover, the shelf life test result proves that the shelf life of the fluorescence detection reagent containing a certain amount of silicone oil or liquid paraffin can reach 6 months. Therefore, the formulation is suitable for mass production and has high industrial practicability.

In a specific example, the irradiation lasts for 10-100 milliseconds; preferably, the irradiation lasts for 50-100 milliseconds.

In a specific example, in step (d), the light intensity for irradiating the ATTO532 is 30-40 milliwatts and the light intensity for irradiating the ATTO647N is 60-80 milliwatts; and/or the ATTO532 or the ATTO647N in a field of view is irradiated for 50-500 milliseconds. Preferably, the ATTO532 or the ATTO647N in a field of view is irradiated for 50-100 milliseconds.

The fluorescence detection reagent of an embodiment of the present disclosure includes an enzymatic oxygen scavenging system and a plurality of reducing agents and no triethylenediamine. In a specific example, the enzymatic oxygen scavenging system is selected from a combination I including glucose and glucose oxidase, a combination II including glucose, glucose oxidase and catalase or a combination III including protocatechuic acid and protocatechuate 3,4-dioxygenase. In a specific example, the plurality of reducing agents are selected from at least two of ascorbic acid, gallic acid, an analog or derivative of gallic acid, cyanuric acid and water-soluble vitamin E. In a specific example, the fluorescence detection reagent includes the combination I, ascorbic acid and gallic acid or the analog or derivative of gallic acid. The fluorescence detection reagent of this embodiment uses glucose and glucose oxidase as an oxygen scavenging system and has a high oxygen removal speed; it also contains ascorbic acid as a reducing agent, fluorescent dye in triplet state is quenched by an electron transfer method and thus its reaction with oxygen molecules is avoided. This is beneficial to the restoration of the fluorescent dye to a ground state, so that an effective and stable detection solution system is well formed, and during fluorescence detection, the fluorescence signal can be stabilized, nucleic acid damage is reduced or inhibited, and the quenching time of the fluorescent molecule is prolonged, thus increasing the sequencing read length and improving the sequencing quality. The combination of ascorbic acid and gallic acid can improve the detected fluorescence signal intensity and imaging quality score, allow the imaging quality to be more stable in the signal acquisition process, and reduce the decreasing amplitude of the imaging quality in the signal acquisition process. The imaging quality refers to the initial analysis obtained by observing a single-molecule image and counting the brightness, the score value and the like through a mini program after introduction of a fluorescence detection reagent.

In a specific example, the fluorescence detection reagent includes 50 mM-200 mM glucose, 2 U/mL-20 U/mL glucose oxidase, 1 mM-200 mM ascorbic acid and 1 mM-10 mM gallic acid or the analog or derivative of gallic acid.

Preferably, in an example, the fluorescence detection reagent includes 80 mM-150 mM glucose, 8 U/mL-12 U/mL glucose oxidase, 1 mM-50 mM ascorbic acid and 1 mM-10 mM gallic acid or the analog or derivative of gallic acid.

In a specific example, the fluorescence detection reagent further includes water-soluble vitamin E (Trolox) and p-benzoquinone. It is found in studies that the water-soluble vitamin E has a protection effect on fluorophore, but the freshly prepared fluorescence detection reagent containing water-soluble vitamin E has poor imaging performance on fluorescent dyes such as Atto532, and the fluorescence detection reagent that has been stored does not have the problem. This may be because that the protection of the fluorophore by the water-soluble vitamin E requires the combined action of Trolox in reducing state and TX-quinone (TQ) in oxidized state. Therefore, it is speculated that most of Trolox in the freshly prepared fluorescence detection reagent is in a reducing state and TQ is not present, and thus a good condition for excitation of the fluorescent dye cannot be created. Based on the above speculation, what is tried is to add a quinone derivative such as p-benzoquinone in a certain proportion to a freshly prepared fluorescence detection reagent; the p-benzoquinone is used for simulating Trolox in an oxidized state, so that the purpose of enhancing the signal intensity of fluorophore is achieved and thereby the imaging quality can be improved, the sequencing read length can be increased, and the error rate can be reduced.

Optionally, the water-soluble vitamin E has a concentration of 6 mM-12 mM, and the p-benzoquinone has a concentration of 0.1 mM-1 mM; preferably, the p-benzoquinone has a concentration of 0.36 mM-0.96 mM.

In a specific example, the fluorescence detection reagent further includes cyanuric acid. Therefore, cyanuric acid is present in the solution in a free form and reacts with oxygen radicals preferentially over molecules to be tested, such as nucleic acids on the surface of a chip, so that the oxygen radicals generated after laser irradiation can be consumed, and thereby damage to nucleic acid chains is reduced or avoided and wrong identification of nucleic acids in sequencing technology can be reduced. In certain examples, the mass percent of cyanuric acid is 0.0001%-0.001%; preferably, the mass percent of cyanuric acid is 0.0003%-0.0009%.

In a specific example, the fluorescence detection reagent further includes at least one of adenosine monophosphate, cytidine monophosphate, guanosine monophosphate, uridine monophosphate and thymidine monophosphate. The inventors guess that similar to cyanuric acid, these components are present in the solution in a free form and react with oxygen radicals preferentially over molecules to be tested, so that the oxygen radicals generated after laser irradiation can be consumed and thereby damage to nucleic acid chains is reduced or avoided and the error rate of sequencing is reduced.

In a specific example, the fluorescence detection reagent further includes 5′-adenosine monophosphate and guanosine-5′-monophosphate. Optionally, the fluorescence detection reagent further includes 1 μM-20 μM 5′-adenosine monophosphate and 1 μM-20 μM guanosine-5′-monophosphate. Preferably, the fluorescence detection reagent further includes 4 μM-12 μM 5′-adenosine monophosphate and 4 μM-12 μM guanosine-5′-monophosphate.

In a specific example, the fluorescence detection reagent uses a Tris (tris(hydroxymethyl)aminomethane) buffer system or a HEPES buffer system; the fluorescence detection reagent has a pH of 6.5-8.5, and preferably, the fluorescence detection reagent has a pH of 8.5. Optionally, the tris(hydroxymethyl)aminomethane or HEPES has a concentration of 80 mM-120 mM. It is found in sequencing that the imaging quality gradually deteriorates as the sequencing progresses, and the data quality in the later stage of sequencing is greatly affected. By detecting the physicochemical properties of the fluorescence detection reagent at different times, it is found that its pH value is gradually reduced, and the dissolved oxygen value is gradually increased. Based on the fact that glucose oxidase and glucose are used as an oxygen scavenging system in the fluorescence detection reagent, it is determined that the reason for the pH reduction is that glucose is oxidized into gluconic acid. In the later stage of sequencing, glucose is depleted and the dissolved oxygen is increased. The pH can be maintained ≥7 for a long time by 4-(2-hydroxyethyl) piperazine-1-ethanesulfonic acid (HEPES) at a certain concentration, so that the stability of the fluorescence detection reagent is improved. In addition, it is further found that the use of tris(hydroxymethyl)aminomethane (Tris) instead of HEPES is more beneficial to the improvement of the stability of the fluorescence detection reagent.

In a specific example, the fluorescence detection reagent further includes acetonitrile, and the volume percent of acetonitrile is 10%-40%; preferably, the volume percent of acetonitrile is 18%-35%; more preferably, the volume percent of acetonitrile is 28%-35%. The combined use of Tris and ACN enables better performance in fluorescence signal intensity, imaging quality and error rate.

In a specific example, the fluorescence detection reagent includes the following components: 50 mM-200 mM glucose, 2 U/mL-20 U/mL glucose oxidase, 1 mM-200 mM ascorbic acid, 1 mM-10 mM gallic acid, 6 mM-12 mM water-soluble vitamin E, 0.1 mM-1.0 mM p-benzoquinone, 0.0001 wt % to 0.001 wt % cyanuric acid, 1 μM-20 μM 5′-adenosine monophosphate, 1 μM-20 μM guanosine-5′-monophosphate, 100 mM-300 mM tris(hydroxymethyl)aminomethane and 18 v/v %-35 v/v % acetonitrile. Optionally, the fluorescence detection reagent further includes a monovalent soluble salt such as sodium chloride, which is in combination with the buffer component.

In a preferred example, the fluorescence detection reagent includes the following components: 80 mM-150 mM glucose, 8 U/mL-12 U/mL glucose oxidase, 1 mM-50 mM ascorbic acid, 1 mM-10 mM gallic acid, 6 mM-12 mM water-soluble vitamin E, 0.36 mM-0.96 mM p-benzoquinone, 0.0003 wt %-0.0009 wt % cyanuric acid, 4 μM-12 μM 5′-adenosine monophosphate, 4 μM-12 μM guanosine-5′-monophosphate, 100 mM-300 mM tris(hydroxymethyl)aminomethane and 18 v/v %-35 v/v % acetonitrile.

The method for preparing the fluorescence detection reagent described above provided according to an embodiment of the present disclosure includes the following step: adding liquid paraffin and/or silicone oil before the fluorescence detection reagent is encapsulated.

The kit provided according to an embodiment of the present disclosure includes the fluorescence detection reagent described above and the nucleotide analog described above. It will be appreciated that the fluorescence detection reagent described above may be used not only for sequencing, but also used in other methods or products where fluorescence detection is desired.

The present disclosure is described in detail below by way of specific examples, and it will be appreciated that the examples are exemplary only. The materials, reagents, sequences, and the like mentioned in the examples can be prepared or synthesized or commercially available, unless otherwise specified.

### Example 10

The fluorescence detection reagents of the experimental groups and the control group were prepared according to the formulations shown in the table below. The experimental group 1 was different from the experimental group 3 in the enzymatic oxygen scavenging system, which was glucose and glucose oxidase in the experimental group 1 and protocatechuate-3,4-dioxygenase (PCD) and 3,4-dihydroxybenzoic acid (protocatechuic acid/PCA) in the experimental group 3; the experimental group 2 was different from the experimental group 1 in that the buffer system HEPES was replaced with Tris. Sequencing was performed using the fluorescence detection reagent of each experimental group, and the fluorescence quenching time of Atto647N-labeled terminator under specific laser working intensity was compared.

group

| | Experimental | | Experimental | | Experimental | |
|---|---|---|---|---|---|---|
| Component | group 1 | | group 2 | | group 3 | |
| Sodium chloride | 20 | mM | 20 | mM | 20 | mM |
| Water-soluble vitamin E | 12 | mM | 12 | mM | 12 | mM |
| Buffer system | HEPES | | MES 25 mM, | | HEPES | |
| | 77 mM, MES 25 mM, | | 100 mM | | 77 mM, MES 25 mM, | |
| | Tris 3 mM | | | | Tris 3 mM | |
| Glucose | 100 | mM | 100 | mM | 0 | |
| Glucose oxidase | 10.4 | U/mL | 10.4 | U/mL | 0 | |
| Acetonitrile | 30.25% | | 30.25% | | 30.25% | |
| Triethylenediamine | 5 | mM | 5 | mM | 5 | mM |
| (DABCO) | | | | | | |

| | | | | |
|---|---|---|---|---|
| PCD | 0 | 0 | 50 | nM |
| PCA | 0 | 0 | 2.5 | mM |

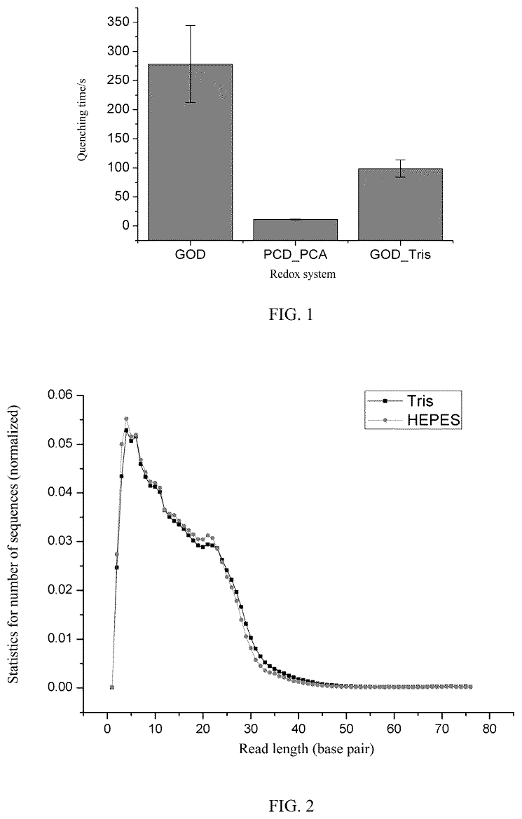

After an oxygen scavenging system was added to the solution, it could be ensured that no oxygen was detected in the test of the dissolved oxygen content in the system using a dissolved oxygen analyzer. However, different systems had different quenching time in actual use. FIG. 1 shows the detection data for different enzymatic oxygen scavenging systems. When the glucose and glucose oxidase oxygen scavenging system was used, the average fluorescence quenching time of Atto647N was 278 s, while when the PCD-PCA oxygen scavenging system was used, the average fluorescence quenching time was 11 s; the times of experiment repetition N=9. The fluorescence quenching time of Atto647N was longer when glucose and glucose oxidase oxygen scavenging system was used. If the exposure time was 0.5 s, 0.2% of the fluorescent molecules would be quenched when the glucose and glucose oxidase oxygen scavenging system was used, while 4.5% of the fluorescent molecules would be quenched when the PCD-PCA oxygen scavenging system was used. Therefore, using glucose and glucose oxidase as the oxygen scavenging system of fluorescence detection reagents can prolong the quenching time of fluorescent molecules.

In addition, HEPES is prone to generate hydrogen peroxide under illumination, so that quenching of fluorescent molecules may be accelerated and photochemical reactions of biomacromolecules may be increased. Therefore, it is more reasonable to replace HEPES with tris(hydroxymethyl)aminomethane (Tris) in principle. Apparent sequencing read lengths under the two different buffer systems were also compared. FIG. 2 shows that there was no significant difference in sequencing read length between experimental group 1 and experimental group 2, and FIG. 1 shows that the average quenching time of experimental group 2 was 99 s. In the case of the extreme exposure time of 0.5 s, 0.6% of the fluorescent molecules were quenched when the fluorescence detection reagent using Tris instead of HEPES was used, and the quenching proportion was small.

### Example 12

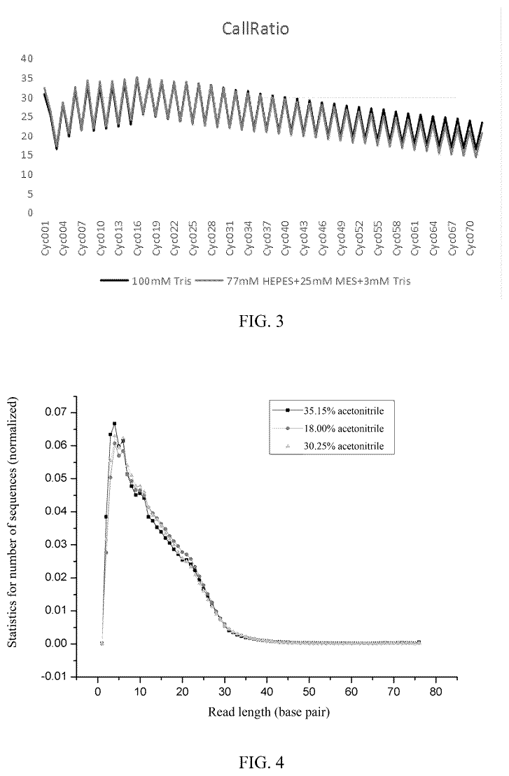

In order to simplify the formulation, the 4-(2-hydroxyethyl) piperazine-1-ethanesulfonic acid (HEPES)+2-(N-morpholine) ethanesulfonic acid (MES)+tris(hydroxymethyl)aminomethane (Tris) combination buffer system in the above formulation was replaced with 100 mM Tris. As shown in FIG. 3, as the sequencing progressed, the CallRatio (CallRatio represents the proportion of bases capable of being identified and can react the sequencing performance of an imaging solution to a certain extent) of 100 mM Tris was gradually better than that of a 77 mM HEPES+25 mM MES+3 mM Tris combination buffer system, which was particularly more obvious in the later stage of sequencing, indicating that 100 mM Tris is more favorable for improving the stability of the performance of a fluorescence detection reagent.

In addition, the performances of Bis-Tris propane, 4-(2-hydroxyethyl) piperazine-1-ethanesulfonic acid (HEPES), tris(hydroxymethyl)aminomethane (Tris) and acetonitrile (ACN) combination plus storage with paraffin or silicone oil and phosphate buffered saline (PBS) were also compared, where the Tris and acetonitrile combination had better performance in fluorescence signal intensity, imaging quality and error rate.

| | Experimental | | Experimental | | Experimental | | Experimental | |
|---|---|---|---|---|---|---|---|---|
| Component | group 1 | | group 2 | | group 3 | | group 4 | |
| Sodium chloride | 20 | mM | 20 | mM | 20 | mM | 20 | mM |
| Water-soluble | 12 | mM | 12 | mM | 12 | mM | 12 | mM |
| vitamin E | | | | | | | | |
| Buffer system | Tris 100 | mM | Tris 100 | mM | 3X | PBS | Bis-tris propane | |
| | | | | | | | 50 mM | |
| Glucose | 100 | mM | 100 | mM | 100 | mM | 100 | mM |
| Glucose oxidase | 10.4 | U/mL | 10.4 | U/mL | 10.4 | U/mL | 10.4 | U/mL |
| Acetonitrile | 0 | | 30.25% | | 0 | | 0 | |
| Triethylenediamine | 5 | mM | 5 | mM | 5 | mM | 5 | mM |
| (DABCO) | | | | | | | | |
| Sodium iodide | 5.25 | mM | 5.25 | mM | 5.25 | mM | 5.25 | mM |

| | | | | | |
|---|---|---|---|---|---|
| Paraffin | 0 | 5 | v/v % | 0 | 0 |

| Experimental | ImageScore_{—} | ImageScore_{—} | Intensity_{—} | Intensity_{—} | |
|---|---|---|---|---|---|
| conditions | Atto532 | Atto647N | Atto532 | Atto647N | Deletion_G |
| Tris + paraffin/ | 3.02 | 2.10 | 169 | 334 | 5.27% |
| silicone oil | | | | | |
| Tris + acetonitrile + | 3.32 | 2.38 | 193 | 383 | 1.87% |
| paraffin/silicone oil | | | | | |
| 3 × PBS | 2.80 | 2.23 | 155 | 318 | 3.58% |
| Bis-tris propane | 2.96 | 2.13 | 175 | 364 | 3.80% |

### Example 14

The fluorescence quantum efficiency of the fluorescent molecule is also influenced by the polarity of the solvent, so acetonitrile at different proportions may cause different fluorescence quantum efficiencies and thus different fluorescence signal intensities are presented. As shown in the table below, in this example, the final concentration of acetonitrile in the fluorescence detection reagent was adjusted and the effect of acetonitrile at different concentrations on sequencing read length, imaging quality and sequencing error rate was compared. The sequencing here involved two fluorescent dyes, Atto532 and Atto647N.

| | Experimental | | Experimental | | Experimental | |
|---|---|---|---|---|---|---|
| Component | group 1 | | group 2 | | group 3 | |
| Sodium chloride | 20 | mM | 20 | mM | 20 | mM |
| Water-soluble | 6 | mM | 6 | mM | 6 | mM |
| vitamin E | | | | | | |
| P-benzoquinone | 0.96 | mM | 0.96 | mM | 0.96 | mM |
| Buffer system | Tris 100 | mM | Tris 100 | mM | Tris 100 | mM |
| Glucose | 100 | mM | 100 | mM | 100 | mM |
| Glucose oxidase | 10.4 | U/mL | 10.4 | U/mL | 10.4 | U/mL |
| Acetonitrile | 30.25% | | 35.15% | | 18% | |
| Triethylenediamine | 5 | mM | 5 | mM | 5 | mM |
| (DABCO) | | | | | | |
| Sodium iodide | 5.25 | mM | 5.25 | mM | 5.25 | mM |
| Ascorbic acid | 50 | mM | 50 | mM | 50 | mM |
| Gallic acid | 20 | mM | 20 | mM | 20 | mM |
| AMP | 8 | uM | 8 | uM | 8 | uM |
| GMP | 8 | uM | 8 | uM | 8 | uM |

| | | | |
|---|---|---|---|
| Cyanuric acid | 0.0003% | 0.0003% | 0.0003% |

As shown in FIG. 4, no significant difference in the apparent read length was caused by acetonitrile at different concentrations in the fluorescence detection reagent, but as shown in the table below, the detected signal intensities were different and the scores of the imaging quality and the error rates were different in the presence of acetonitrile at different concentrations in the fluorescence detection reagent.

| Experimental | ImageScore_{—} | ImageScore_{—} | Intensity_{—} | Intensity_{—} | Error |
|---|---|---|---|---|---|
| conditions | Atto532 | Atto647N | Atto532 | Atto647N | Rate |
| 18% acetonitrile | 3.25 | 2.31 | 172 | 333 | 5.51% |
| 30.25% acetonitrile | 3.26 | 2.45 | 173 | 348 | 4.83% |
| 35.15% acetonitrile | 3.32 | 2.38 | 193 | 383 | 4.76% |

Therefore, experiments prove that the acetonitrile within the concentration range of 10-40%, particularly within the range of 28-35%, can result in good sequencing read length, where the fluorescence detection reagent containing 30% acetonitrile has better performance in the signal intensity, imaging quality score and error rate.

### Example 16

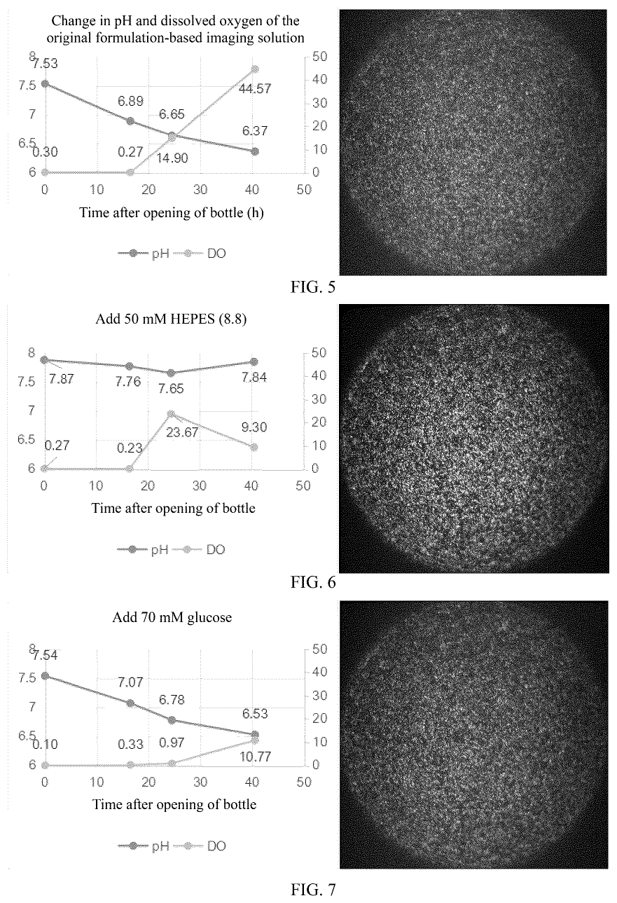

As shown in FIG. 5, it was found in sequencing that the imaging quality gradually deteriorated as the sequencing progressed, and the data quality in the later stage of sequencing was greatly affected. By detecting the physicochemical properties of the fluorescence detection reagent at different times, it was found that its pH value was gradually reduced, and the dissolved oxygen value was gradually increased. Based on the fact that glucose oxidase and glucose were used as an oxygen scavenging system, it was determined that the reason for the pH reduction was that glucose was oxidized into gluconic acid. In the later stage of sequencing, glucose was depleted and the dissolved oxygen was increased.

Therefore, the following experiment was performed to increase the stability of the fluorescence detection reagent. 25 mL of imaging solution was prepared, placed in a kit and left to stand at 4° C. for 40 h, and the pH and dissolved oxygen of the solution were measured at different time points, with the variables being the concentration of glucose and the concentration of HEPES (pH 8.8). The specific scheme is shown in the table below.

| | Control | | Experimental | | Experimental | | Experimental | |
|---|---|---|---|---|---|---|---|---|
| Component | group | | group 1 | | group 2 | | group 3 | |
| Water-soluble | 12 | mM | 12 | mM | 12 | mM | 12 | mM |
| vitamin E | | | | | | | | |
| Buffer system | HEPES | | HEPES | | HEPES | | HEPES | |
| | 27 mM, | | 77 mM, | | 27 mM, | | 77 mM, | |
| | MES 25 mM, | | MES 25 mM, | | MES 25 mM, | | MES 25 mM, | |
| | Tris 3 mM | | Tris 3 mM | | Tris 3 mM | | Tris 3 mM | |
| Glucose | 30 | mM | 30 | mM | 100 | mM | 100 | mM |
| Glucose oxidase | 10.4 | U/mL | 10.4 | U/mL | 10.4 | U/mL | 10.4 | U/mL |
| Acetonitrile | 30.25% | | 30.25% | | 30.25% | | 30.25% | |
| Triethylenediamine | 5 | mM | 5 | mM | 5 | mM | 5 | mM |
| (DABCO) | | | | | | | | |
| Sodium iodide | 5.25 | mM | 5.25 | mM | 5.25 | mM | 5.25 | mM |

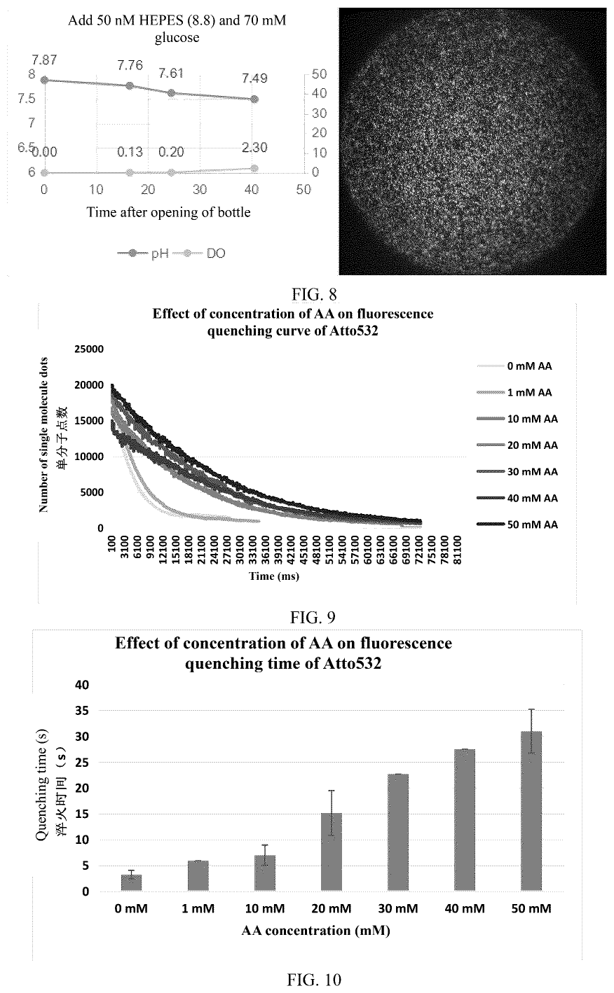

As shown in FIGS. 5-8, increasing the concentration of HEPES (pH 8.8) could maintain the pH ≥7 for 40 h, and increasing the concentration of glucose could slow down the rate of increase of dissolved oxygen in the fluorescence detection reagent. Therefore, the HEPES (pH8.8) and the glucose being in a concentration range of 80-150 mM exhibits better stabilizing effect on the fluorescence detection reagent.

### Example 18

The fluorescence detection reagents of all the groups were prepared according to the formulations shown in the table below, and the difference between these groups was that ascorbic acid (AA) was added at different concentrations or no ascorbic acid was added. Sequencing was performed using the fluorescence detection reagent of each group, and the fluorescence quenching time of Atto532-labeled terminator under laser working intensity was compared.

| | Control | | Experimental | | Experimental | | Experimental | | Experimental | | Experimental | | Experimental | |
|---|---|---|---|---|---|---|---|---|---|---|---|---|---|---|
| Component | group | | group 1 | | group 2 | | group 3 | | group 4 | | group 5 | | group 6 | |
| Water-soluble | 12 | mM | 12 | mM | 12 | mM | 12 | mM | 12 | mM | 12 | mM | 12 | mM |
| vitamin E | | | | | | | | | | | | | | |
| Buffer system | Tris 100 | | Tris 100 | | Tris 100 | | Tris 100 | | Tris 100 | | Tris 100 | | Tris 100 | |
| | mM | | mM | | mM | | mM | | mM | | mM | | mM | |
| Glucose | 100 | mM | 100 | mM | 100 | mM | 100 | mM | 100 | mM | 100 | mM | 100 | mM |
| Glucose oxidase | 10.4 | U/mL | 10.4 | U/mL | 10.4 | U/mL | 10.4 | U/mL | 10.4 | U/mL | 10.4 | U/mL | 10.4 | U/mL |
| Acetonitrile | 30.25% | | 30.25% | | 30.25% | | 30.25% | | 30.25% | | 30.25% | | 30.25% | |
| Triethylenediamine | 5 | mM | 5 | mM | 5 | mM | 5 | mM | 5 | mM | 5 | mM | 5 | mM |
| (DABCO) | | | | | | | | | | | | | | |
| Sodium iodide | 5.25 | mM | 5.25 | mM | 5.25 | mM | 5.25 | mM | 5.25 | mM | 5.25 | mM | 5.25 | mM |

| | | | | | | | | | | | | | |
|---|---|---|---|---|---|---|---|---|---|---|---|---|---|
| Ascorbic acid | 0 | 1 | mM | 10 | mM | 20 | mM | 30 | mM | 40 | mM | 50 | mM |

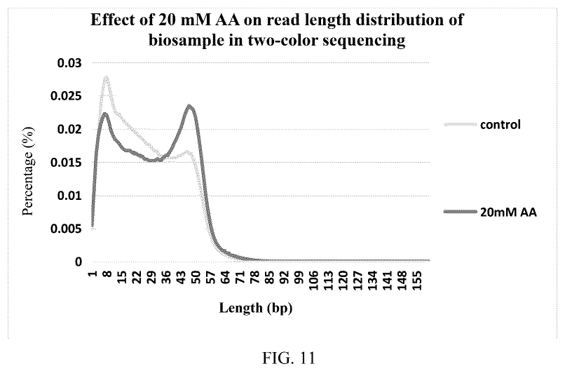

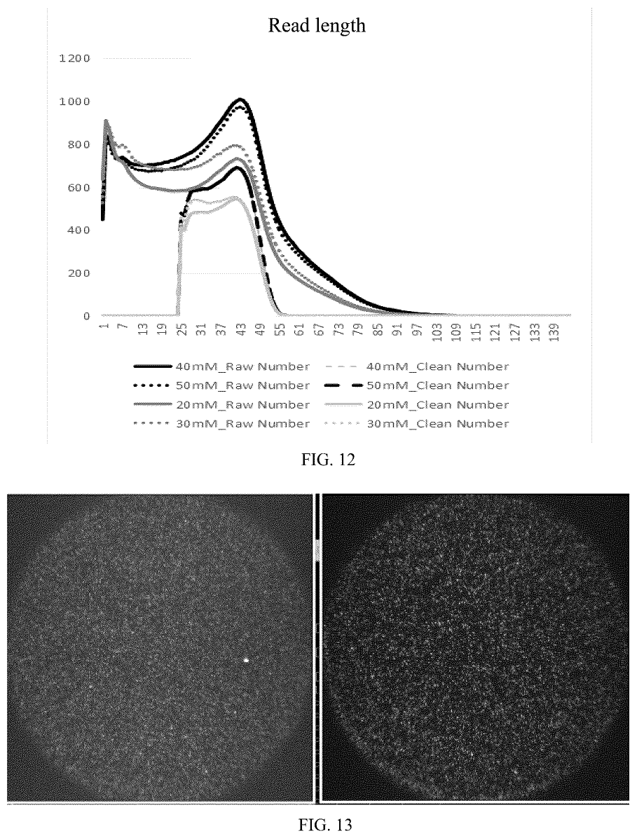

FIG. 9 shows the quenching curves of Atto532 after addition of AA at different concentrations, and it can be seen from the graph that the quenching curve decreased more slowly with the increase of AA concentration, and the quenching curve of 50 mM AA decreased the slowest. According to the quenching time in FIG. 10, it can be seen that the quenching time of the control group without AA was within 5 s, while the quenching time was prolonged after addition of AA, and the quenching time of the 50 mM AA group reached 30 s with the increase of AA concentration, i.e., AA helps to prolong the quenching time of Atto532. Furthermore, as the concentration of AA increases, the sequencing read length increases. The distribution of the sequencing read lengths of the 20 mM AA group and the control group (0 mM AA) is shown in FIG. 11, and for the read length peak, the main peak was higher and the short fragment was reduced after 20 mM AA was added. FIG. 12 shows the read length distribution of groups containing ascorbic acid at different concentrations, and it can be seen that the 40 mM AA group and the 50 mM AA group were in better concentration ranges, with the read length increased by 1 bp, the main peak more prominent and the small fragments significantly reduced. From the above studies, it can be seen that the stability of the system can be further increased as the concentration of ascorbic acid is increased. The above results prove that the addition of AA is helpful for prolonging the quenching time of fluorescent molecules and increasing the sequencing read length, and the concentration range of 40 mM-50 mM is better.

It is found in other experimental tests that the concentration of AA in the solution being 100-200 mM also can further reduce the quenching time (data not shown here).

### Example 20

Fluorescence detection reagents of an experimental group and a control group were prepared, and the difference between the two groups was that both ascorbic acid and gallic acid were added to the experimental group and only ascorbic acid but no gallic acid (GA) was added to the control group. Sequencing was performed using the fluorescence detection reagents of the experimental group and the control group, and the fluorescence signal intensities, imaging quality scores and other data were compared.

| | | |
|---|---|---|
| | Control | Experimental |
| Component | group | group |

| | | | | |
|---|---|---|---|---|
| Sodium chloride | 20 | mM | 20 | mM |
| Water-soluble | 6 | mM | 6 | mM |
| vitamin E | | | | |
| P-benzoquinone | 0.96 | mM | 0.96 | mM |
| Buffer system | Tris 100 | mM | Tris 100 | mM |
| Glucose | 100 | mM | 100 | mM |
| Glucose oxidase | 10.4 | U/mL | 10.4 | U/mL |

| | | |
|---|---|---|
| Acetonitrile | 30.25% | 30.25% |

| | | | | |
|---|---|---|---|---|
| Triethylenediamine | 5 | mM | 5 | mM |
| (DABCO) | | | | |
| Sodium iodide | 5.25 | mM | 5.25 | mM |
| Ascorbic acid | 50 | mM | 50 | mM |
| Gallic acid | 0 | | 20 | mM |
| AMP | 8 | μM | 8 | μM |
| GMP | 8 | μM | 8 | μM |

| | | |
|---|---|---|
| Cyanuric acid | 0.0003% | 0.0003% |

The AA gradient assay showed that the quenching time of Atto532 increased significantly with increasing concentration of the reducing agent AA, but the fluorescence signal intensity tended to decrease with increasing AA concentration. On the basis of AA, the combination test in combination with GA was performed and the results are shown in the table below. The results show that the combination of ascorbic acid and gallic acid can improve the detected fluorescence signal intensity and imaging quality score, allow the imaging quality to be more stable in the signal acquisition process, and reduce the decreasing amplitude of the imaging quality in the signal acquisition process. Therefore, the addition of gallic acid in the presence of ascorbic acid can enhance the stability of the imaging quality.

| | | | | | | | Imaging | Imaging | |
|---|---|---|---|---|---|---|---|---|---|
| | | | | | | | quality | quality | |
| | | | Signal | Signal | Imaging | Imaging | score | score | |
| | Signal | Signal | decreasing | decreasing | quality | quality | decreasing | decreasing | |
| Experimental | intensity_{—} | intensity_{—} | amplitude_{—} | amplitude_{—} | score_{—} | score_{—} | amplitude_{—} | amplitude_{—} | Error |
| conditions | Atto532 | Atto647N | Atto532 | Atto647N | Atto532 | Atto647N | Atto532 | Atto647N | rate |
| Ascorbic | 213 | 399 | 18 | 19 | 3.19 | 2.38 | 0.26 | −0.01 | 5.41% |
| acid + gallic | | | | | | | | | |
| acid | | | | | | | | | |
| Ascorbic | 208 | 393 | 22 | 23 | 3.09 | 2.27 | 0.5 | 0.21 | 5.64% |
| acid | | | | | | | | | |

### Example 22

The fluorescence detection reagents of the experimental groups and the control group were prepared according to the formulations shown in the table below, and the difference between the experimental groups and the control group was that both water-soluble vitamin E and p-benzoquinone at different concentrations were added to each experimental group and only water-soluble vitamin E but no p-benzoquinone was added to the control group. Sequencing was performed using the fluorescence detection reagents of the experimental groups and the control group, and the imaging quality, read lengths, error rates and other data were compared.

| | Experimental | Experimental | Experimental | Experimental | Experimental | Experimental | Experimental | Experimental | Control |
|---|---|---|---|---|---|---|---|---|---|
| Component | group 1 | group 2 | group 3 | group 4 | group 5 | group 6 | group 7 | group 8 | group |
| Sodium | 20 | 20 | 20 | 20 | 20 | 20 | 20 | 20 | 20 |
| chloride | mM | mM | mM | mM | mM | mM | mM | mM | mM |
| Ascorbic | 40 | 40 | 40 | 40 | 40 | 40 | 40 | 40 | 40 |
| acid | mM | mM | mM | mM | mM | mM | mM | mM | mM |
| Gallic | 5 | 5 | 5 | 5 | 5 | 5 | 5 | 5 | 5 |
| acid | mM | mM | mM | mM | mM | mM | mM | mM | mM |
| Cyanuric | 0.0003% | 0.0003% | 0.0003% | 0.0003% | 0.0003% | 0.0003% | 0.0003% | 0.0003% | 0.0003% |
| acid | | | | | | | | | |
| 5′-adenosine | 0.008 | 0.008 | 0.008 | 0.008 | 0.008 | 0.008 | 0.008 | 0.008 | 0.008 |
| monophosphate | mM | mM | mM | mM | mM | mM | mM | mM | mM |
| Guanosine-5′- | 0.008 | 0.008 | 0.008 | 0.008 | 0.008 | 0.008 | 0.008 | 0.008 | 0.008 |
| monophosphate | mM | mM | mM | mM | mM | mM | mM | mM | mM |
| Water-soluble | 12 | 12 | 6 | 6 | 6 | 3 | 3 | 3 | 12 |
| vitamin E | mM | mM | mM | mM | mM | mM | mM | mM | mM |
| P- | 0.6 | 0.36 | 0.96 | 0.6 | 0.36 | 0.6 | 0.36 | 0.18 | 0 |
| benzoquinone | mM | mM | mM | mM | mM | mM | mM | mM | mM |
| Tris | 200.176 | 200.176 | 200.176 | 200.176 | 200.176 | 200.176 | 200.176 | 200.176 | 200.176 |
| | mM | mM | mM | mM | mM | mM | mM | mM | mM |
| Glucose | 130 | 130 | 130 | 130 | 130 | 130 | 130 | 130 | 130 |
| | mM | mM | mM | mM | mM | mM | mM | mM | mM |
| Glucose | 10.4 | 10.4 | 10.4 | 10.4 | 10.4 | 10.4 | 10.4 | 10.4 | 10.4 |
| oxidase | U/mL | U/mL | U/mL | U/mL | U/mL | U/mL | U/mL | U/mL | U/mL |
| Acetonitrile | 0 | 0 | 0 | 0 | 0 | 0 | 0 | 0 | 0 |

As shown in FIG. 13, the fluorescence detection reagent of the experimental group 1 (right panel) was significantly better than the fluorescence detection reagent of the control group (left panel) in terms of the imaging quality of Atto532. As shown in the table below, from the comprehensive evaluation of imaging quality score, read length and error rate, the combination of 6-12 mM water-soluble vitamin E and 0.36-0.96 mM p-benzoquinone can obtain better imaging quality, read length and relatively low error rate, and the combination of 6 mM water-soluble vitamin E and 0.96 mM p-benzoquinone is the optimal condition. Therefore, experiments prove that the combination of water-soluble vitamin E and p-benzoquinone is beneficial to improving the imaging quality and sequencing read length and reducing error rate, and the combination of 6-12 mM water-soluble vitamin E and 0.36-0.96 mM p-benzoquinone is a better concentration range.

| Experimental | Imaging quality | Imaging quality | Read | Error |
|---|---|---|---|---|
| conditions | score_CT | score_GA | length | rate |
| 12 mM Trolox + | 3.20 | 2.43 | 41.16 | 5.97% |
| 0.6 mM BQ | | | | |
| 12 mM Trolox + | 2.38 | 2.32 | 41.87 | 6.77% |
| 0.36 mM BQ | | | | |
| 6 mM Trolox + | 3.38 | 2.43 | 41.34 | 5.53% |
| 0.96 mM BQ | | | | |
| 6 mM Trolox + | 3.24 | 2.43 | 41.4 | 5.63% |
| 0.6 mM BQ | | | | |
| 6 mM Trolox + | 2.87 | 2.33 | 41.51 | 5.82% |
| 0.36 mM BQ | | | | |
| 3 mM Trolox + | 2.40 | 2.25 | 40.87 | 7.52% |
| 0.6 mM BQ | | | | |
| 3 mM Trolox + | 2.35 | 2.25 | 40.99 | 6.90% |
| 0.36 mM BQ | | | | |
| 3 mM Trolox + | 2.26 | 2.15 | 41.71 | 7.33% |
| 0.18 mM BQ | | | | |

### Example 24

The fluorescence detection reagents of all the groups were prepared according to the formulations shown in the table below, and the difference between these groups was that cyanuric acid was added in different proportions or no cyanuric acid was added. Sequencing was performed using the fluorescence detection reagents of the groups, and the error rates were compared.

| | Control | | Experimental | | Experimental | | Experimental | |
|---|---|---|---|---|---|---|---|---|
| Component | group | | group 1 | | group 2 | | group 3 | |
| Sodium chloride | 20 | mM | 20 | mM | 20 | mM | 20 | mM |
| Water-soluble | 12 | mM | 12 | mM | 12 | mM | 12 | mM |
| vitamin E | | | | | | | | |
| Buffer system | Tris 100 | mM | Tris 100 | mM | Tris 100 | mM | Tris 100 | mM |
| Glucose | 100 | mM | 100 | mM | 100 | mM | 100 | mM |
| Glucose oxidase | 10.4 | U/mL | 10.4 | U/mL | 10.4 | U/mL | 10.4 | U/mL |
| Acetonitrile | 30.25% | | 30.25% | | 30.25% | | 30.25% | |
| Triethylenediamine | 5 | mM | 5 | mM | 5 | mM | 5 | mM |
| (DABCO) | | | | | | | | |
| Sodium iodide | 5.25 | mM | 5.25 | mM | 5.25 | mM | 5.25 | mM |
| Ascorbic acid | 50 | mM | 50 | mM | 50 | mM | 50 | mM |
| AMP | 8 | μM | 8 | μM | 8 | μM | 8 | μM |
| GMP | 8 | μM | 8 | μM | 8 | μM | 8 | μM |

| | | | | |
|---|---|---|---|---|
| Cyanuric acid | 0 | 0.0003% | 0.0006% | 0.0009% |

The results are shown in the table below. Adding cyanuric acid in different proportions to the fluorescence detection reagent helps to reduce the error rate of sequencing. Therefore, experiments prove that the error rate can be significantly reduced by adding cyanuric acid, and a better effect can be achieved by adding cyanuric acid at the concentration range of 0.0001%-0.001%. The inventors guess that cyanuric acid is present in the solution in a free form and can react with oxygen radicals preferentially over nucleic acids on the surface of a chip, so that the oxygen radicals generated after laser irradiation are consumed, and thereby damage to nucleic acid chains is reduced or avoided and wrong identification of nucleic acids in sequencing technology can be reduced.

| Experimental conditions | Error rate |
|---|---|
| Cyanuric acid not added | 6.53% |
| 0.0005% cyanuric acid | 6.25% |
| 0.0001% cyanuric acid | 6.26% |
| 0.001% cyanuric acid | 6.23% |

### Example 26

The fluorescence detection reagents of the experimental groups and the control group were prepared according to the formulations shown in the table below, and the difference between the experimental groups and the control group was that 5′-adenosine monophosphate (AMP) and guanosine-5′-monophosphate (GMP) at different concentrations were added to each experimental group and no 5′-adenosine monophosphate (AMP) and guanosine-5′-monophosphate (GMP) was added to the control group. Sequencing was performed using the fluorescence detection reagents of each experimental group and the control group, and the error rates were compared.

| | Control | | Experimental | | Experimental | | Experimental | |
|---|---|---|---|---|---|---|---|---|
| Component | group | | group 1 | | group 2 | | group 3 | |
| Sodium chloride | 20 | mM | 20 | mM | 20 | mM | 20 | mM |
| Water-soluble | 12 | mM | 12 | mM | 12 | mM | 12 | mM |
| vitamin E | | | | | | | | |
| Buffer system | Tris 100 | mM | Tris 100 | mM | Tris 100 | mM | Tris 100 | mM |
| Glucose | 100 | mM | 100 | mM | 100 | mM | 100 | mM |
| Glucose oxidase | 10.4 | U/mL | 10.4 | U/mL | 10.4 | U/mL | 10.4 | U/mL |
| Acetonitrile | 30.25% | | 30.25% | | 30.25% | | 30.25% | |
| Triethylenediamine | 5 | mM | 5 | mM | 5 | mM | 5 | mM |
| (DABCO) | | | | | | | | |
| Sodium iodide | 5.25 | mM | 5.25 | mM | 5.25 | mM | 5.25 | mM |
| Ascorbic acid | 50 | mM | 50 | mM | 50 | mM | 50 | mM |

| | | | | | | | |
|---|---|---|---|---|---|---|---|
| AMP | 0 | 4 | μM | 8 | μM | 12 | μM |
| GMP | 0 | 4 | μM | 8 | μM | 12 | μM |

The results are shown in the table below. Adding monophosphate at different concentrations to the fluorescence detection reagent helps to reduce the error rate of sequencing. Therefore, experiments prove that the error rate can be reduced by adding the combination of 5′-adenosine monophosphate (AMP) and guanosine-5′-monophosphate (GMP), and the effect is remarkable when the concentration is 1 μM-20 μM. The inventors guess that monophosphate is present in the solution in a free form and can react with oxygen radicals preferentially over nucleic acids on the surface of a chip, so that the oxygen radicals generated after laser irradiation are consumed, and thereby damage to nucleic acid chains is reduced or avoided and wrong identification of bases can be reduced. In other words, unreactive nucleotide analogs can be added as sacrificial or competitive reagents to replace or protect the target reaction substrate nucleotides.

| Experimental conditions | Error rate |
|---|---|
| Not added | 6.81% |
| 1 μM AMP + 1 μM GMP | 6.78% |
| 10 μM AMP + 10 μM GMP | 6.53% |
| 20 μM AMP + 20 μM GMP | 6.53% |

### Example 28

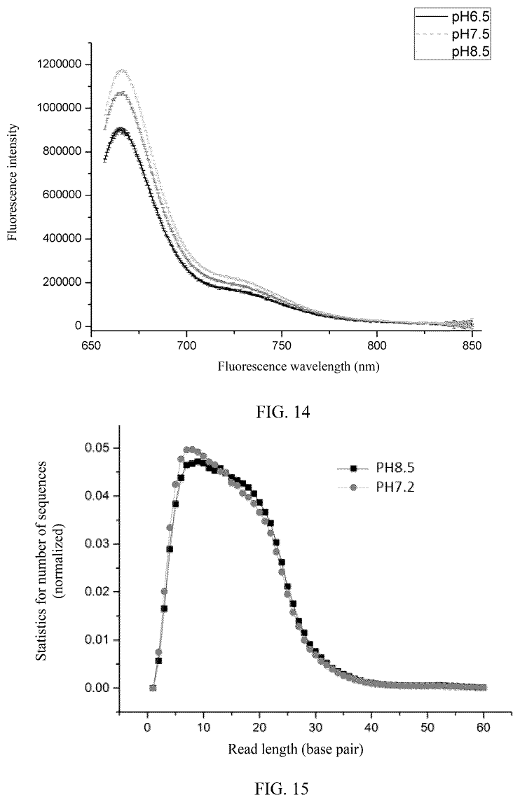

Since the pH can affect the electron transfer of the fluorescent molecule, different pH values may result in different fluorescence quantum efficiencies of the fluorescent molecule and thereby result in different fluorescence brightness. In this example, Atto647N-labeled G terminator was dissolved in Tris solutions with different pH values, and the different solutions were each placed in a quartz chamber, excited by 640 nm laser with constant light intensity, and tested for fluorescence intensity. As shown in FIG. 14, in the pH range of 6.5-8.5, the higher the pH value, the higher the fluorescence intensity of Atto647N. The apparent sequencing read lengths corresponding to the fluorescence detection reagents with and without pH increased were compared through sequencing. As shown in FIG. 15, the sequencing read length corresponding to the fluorescence detection reagent at pH 8.5 was slightly increased, but the superiority was not significant. However, it is recommended to raise the pH of the fluorescence detection reagent to 8.5, considering that the oxygen scavenging system will cause a further decrease in the pH of the solution during oxygen scavenging and thereby affect the oxygen scavenging rate. Therefore, experiments prove that when the pH of the fluorescence detection reagent is within the range of 6.5-8.5, higher pH value results in higher fluorescence intensity of Atto647N and is more beneficial to the stability of the oxygen scavenging system.

### Example 30

In this example, the effect of other additives on the performance of the fluorescence detection reagent was further detected, and the specific steps are as follows.

### 1. Quenching Curve

The quenching curve reflects the quenching of the fluorescent dye of one base; the faster the quenching curve decreases, the shorter the quenching time is, and the worse the stability of the fluorescent dye in an imaging system is; otherwise, the better the stability is; the quenching time is defined as the time corresponding to the decrease of the number of dots (fluorescence signals) to 50% due to quenching.

### 1.1. Selection Principle for Target Sequence

The target sequence of the base to be tested in the first cycle of reaction was selected to be used as a target to be tested (a template molecule with sequence known);

### 1.2. Principles of Data Analysis

A. The number of dots (the fluorescence signal appears as a peak or spot on the image) in each picture is counted; for example, a fixed-size image area 1024*1024 or 512*512 could be selected for counting;
B. The scatter diagram showing the change in the number of dots along with the time is analyzed, and the fitting analysis and statistics of the quenching time were preformed by using the origin software.

### 1.3. Test Process

Assuming that the base to be tested is C_Atto532 (dCTP with Atto532), the test was performed by selecting a single target whose target sequence was G in the first cycle: C_Atto532 base was added according to the common single-molecule sequencing by synthesis process, and under the condition that constant laser intensity was ensured, a fixed area was selected and a photo was acquired every 100 ms until all fluorescence signals were completely quenched;

### 2. Long Sequencing Validation

The influence of the additives obtained by screening based on quenching curves on the sequencing quality was verified through two-channel sequencing (two-color sequencing), and the test process is as follows: base polymerization/extension-photograph acquisition in the presence of fluorescence detection reagent-cropping, and this process was repeated. 60 cycles of synthetic target sequencing or 80 cycles of biological sample sequencing were performed.

### 3. Specific Test

Substrate (which is a modified/engineered nucleotide, also known as a reversible terminator, and is a nucleotide that carries a fluorescent molecule and can inhibit the binding of other nucleotides to the next position of the template to be tested) configuration: 1 mL of each of 50-150 nM, e.g., 125 nM, C_Atto647N (dCTP carrying Atto647N), 50-150 nM, e.g., 125 nM, T_Atto647N (dTTP carrying Atto647N), 100-200 nM, e.g., 200 nM, A_Atto647N (dATP carrying Atto647N), 50-200 nM, e.g., 75 nM, G_Atto647N (dGTP carrying Atto647N), 1000 nM T_Atto532 (dTTP carrying Atto532), 1000 nM C_Atto532 (dCTP carrying Atto532), AT mix (A_Atto647N and T_Atto532), and CG mix (G_Atto647N and C_Atto532) was prepared, and the specific preparation is shown in the table below. The specific test volume was adjusted based on experiments.

| | | Nucleotide | |
|---|---|---|---|
| | | analog/base | |
| Serial | Name of | volume | Klenow |
| number | reagent | (μL) | fragment |
| 1 | C_Atto647N | 0.25 | 25 U |
| 2 | C_Atto532 | 2 | 25 U |
| 3 | T_Atto647N | 0.25 | 25 U |
| 4 | A_Atto647N | 0.4 | 25 U |
| 5 | G_Atto647N | 0.15 | 25 U |
| 6 | AT mix | A: 0.4, T: 2 | 50 U |
| 7 | CG mix | G: 0.15, C: 2 | 50 U |

The fluorescence detection solution used contained components in the two tables below:

| Component | | |
|---|---|---|
| Glucose | 70 | mM |
| Glucose | 10.4 | U/mL |
| oxidase | | |

| | |
|---|---|
| BME (mercaptoethanol) | 1 mM, 10 mM, 25 mM, 50 mM |
| DTT (dithiothreitol) | 1 mM, 10 mM, 50 mM |
| AA (ascorbic acid) + MV | In the case of 20 mM AA, the ratio of |
| (methyl viologen) | AA to MV was 1:0.01, 1:0.05, 1:0.1, |
| | 1:0.5, 1:1, 1:3, 1:5, 1:10, or 1:20 |

A single-target sequence of C base in the first cycle of reaction (the binding positions of the base in the first cycle of reaction are shown as the underlined bases in the table) was selected, and the target sequence carried 3′-fam. Reference may be made to the table below for specific sequence. The single-target sequence, target sequence, target, etc., as used herein, refer to a template; the C target in the table below refers to the template preset for the C base (C nucleotide analog added) sequence in the first cycle of sequencing.

| | Sequence (5′-3′) |
|---|---|
| C | CTAGACTGGACTTGACGACTTGCACGTAGCTAGCTGGGTGCGG |
| tar- | AAGAGAAAGAATACCATGCAGAAGGAGGCAAAGTA (SEQ ID |
| get | NO: 1) |
| | TGCAGCTAATGCATGCGCATCTAGAGTCGTACGCTGGGTGCGG |
| | AAGAGAAAGAATACCATGCAGAAGGAGGCAAAGTA (SEQ ID |
| | NO: 2) |
| | TGACGTACCTAGACTGAGTCACGTTCGACGATGCTGGGTGCGG |
| | AAGAGAAAGAATACCATGCAGAAGGAGGCAAAGTA (SEQ ID |
| | NO: 3) |
| | TCATCTATTATGCCATCTCCCTTGTGGAATCAGCTGGGTGCGG |
| | AAGAGAAAGAATACCATGCAGAAGGAGGCAAAGTA (SEQ ID |
| | NO: 4) |

The target sequence was attached to a designated surface/chip by hybridizing with the probe on the surface, C_Atto647N and C_Atto532 were each added for detection, and after the base extension reaction, each fluorescence detection reagent was introduced to the designated surface and image acquisition on the surface was performed, where the laser exposure of C_Atto647N was set to be 100 ms or 500 ms (laser: 60 mW), and the C_Atto532 exposure was set to be 100 ms (laser: 30 mW); thus, different exposure time was selected according to different test bases to continuously acquire photos until the dots were almost quenched, and finally the fitting analysis was performed by using the origin software.

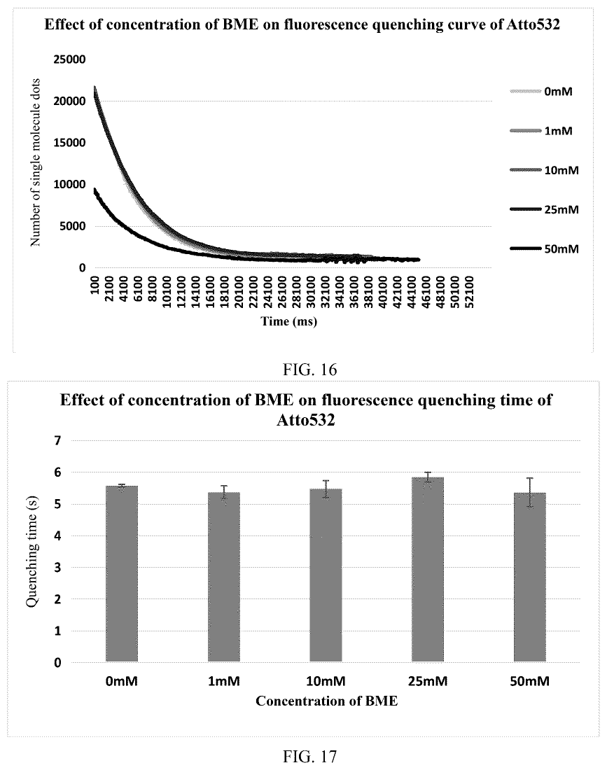

Results: FIG. 16 shows the change in the quenching curves of Atto532 in the presence of BME at different concentrations. As can be seen from the graph, the quenching curves of 1 mM BME, 10 mM BME and 25 mM BME were not significantly different from the quenching curve without BME; the initial dot number of 50 mM BME was significantly lower than that of other experimental groups, but it is unlikely to be one-fold difference in the hybridization density under the same concentration and the same conditions, and it is speculated that BME has cleaving effect on the disulfide bond of the base linker and thereby causes lower initial dot number considering the structure of the base linker and the characteristics of BME. According to FIG. 17, it can be seen that the quenching time of Atto532 was not significantly changed in the presence of BME at different concentrations.

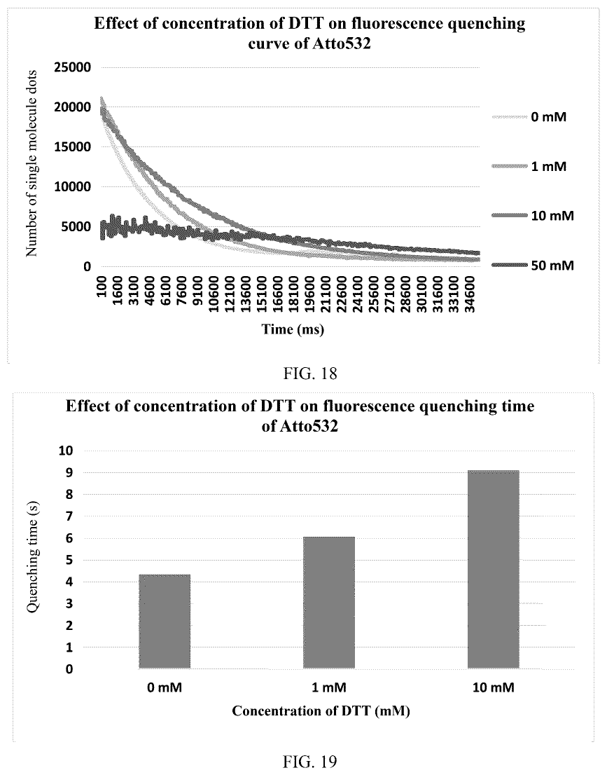

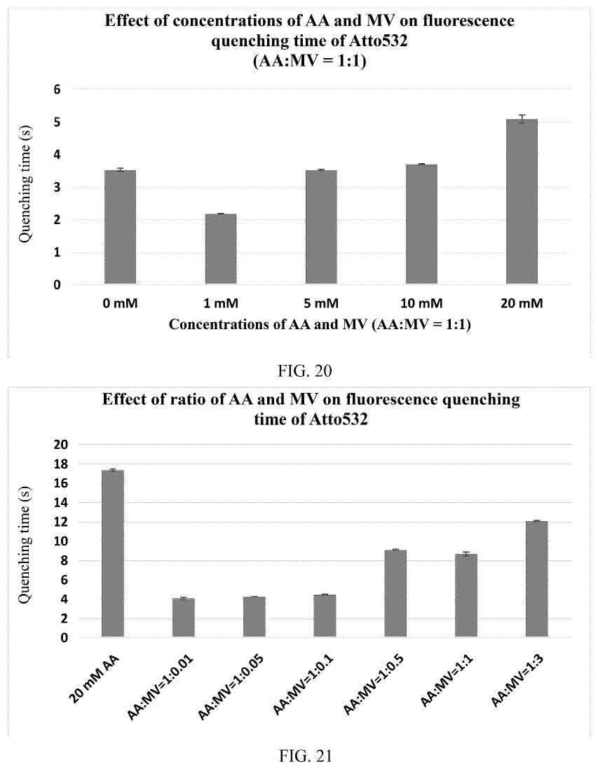

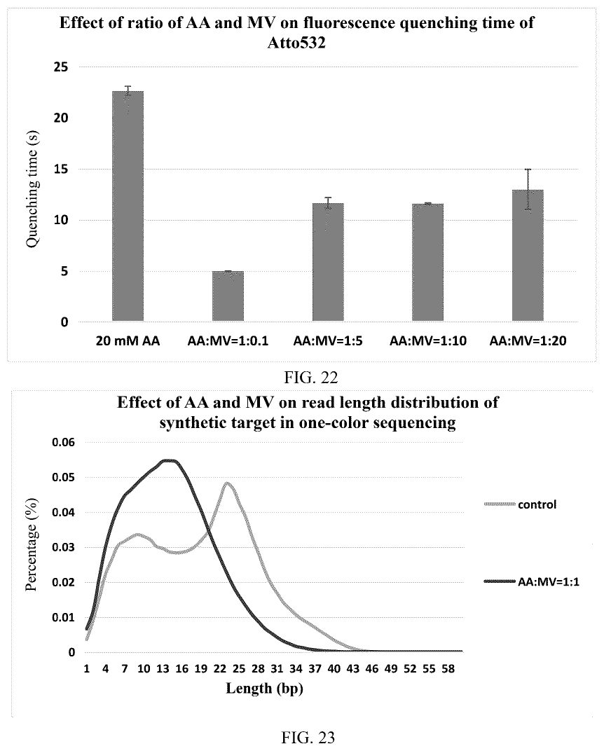

FIG. 18 shows the quenching of Atto532 in the presence of DTT at different concentrations. It can be seen from the quenching graph that the quenching curves decreased more slowly for 1 mM and 10 mM DTT compared with that of the control group without DTT, but DTT and BME had similar functions; the 50 mM (high concentration) DTT had significant cleaving effect on the disulfide bond, and the initial dot number was significantly lower than that of the other test groups. The quenching time was counted for the quenching in the presence of 1 mM and 10 mM DDT, as shown in FIG. 19. As can be seen from the figure, the quenching time significantly increased along with the increase of the concentration of DTT, and in the presence of 10 mM DTT, the quenching time was doubled; that is, DTT can significantly improve the quenching time of Atto532, but the application is difficult because of its cleaving effect on the base linker. For the AA test, a combination system of AA and MV was tried. In FIG. 20, under the condition of same ratio (AA:MV=1:1), the quenching time was improved to some extent relative to the control group without addition of AA and MV as the concentration of AA and MV increased, and the quenching time of Atto532 was extended to 5 s when the concentration was 20 mM (AA:MV=1:1); in addition, the gradient experiment was performed for the ratio of AA to MV; from the comparison with the control group 20 mM AA and the two column charts in FIGS. 21 and 22, it can be seen that the quenching time of Atto532 was shortened when low concentration MV was added in the presence of 20 mM AA, and the quenching time could reach 10-15 s at most with the increase of MV concentration but was still significantly lower than that of the control group 20 mM AA, that is, for Atto532, the addition of MV in the presence of AA is not beneficial to prolonging the quenching time.

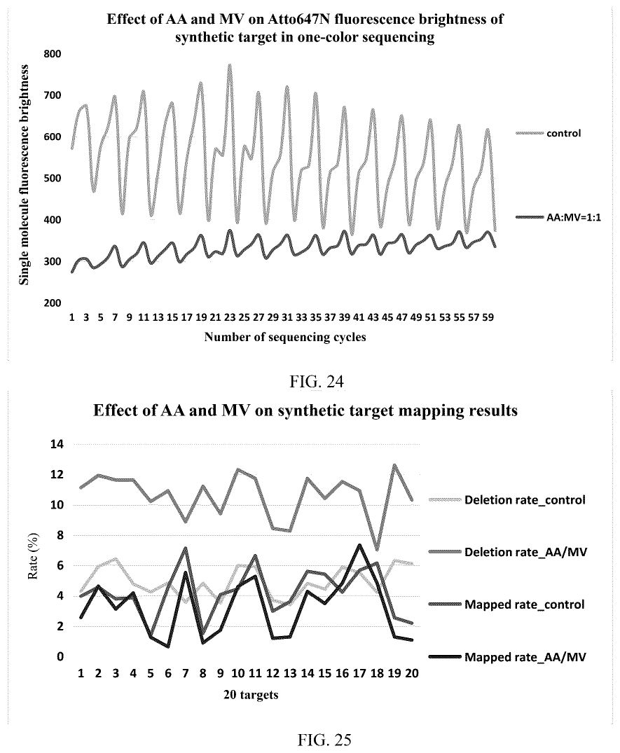

FIGS. 23 and 24 show the test results of the AA and MV combination system. The read length distribution graph 23 shows that, after the addition of 20 mM AA and 20 mM MV (AA:MV=1:1), the main peak of the read length shifted to the left, the sequencing read length became shorter, and the single molecule fluorescence brightness was only about 300, which was significantly lower than that of the control group. This results in a high deletion rate (rate of unidentified bases), as shown in the mapping result in FIG. 25, and the mapped rate was low, and the overall sequencing quality was poor. Therefore, the quality of one-color sequencing cannot be significantly influenced if AA is added alone, while the deletion rate significantly increases if AA is added in combination with MV, the read length is shifted to the left (which indicates that the read length is shortened), the sequencing quality is poor, and the reagent cannot be used.

### Example 32

After the introduction of various antioxidants, the effect of the antioxidant DABCO in the original formulation was unknown, and the effect of sodium iodide as the catalyst of the GOD oxygen scavenging system was also unknown. Therefore, we tried to remove DABCO and sodium iodide in the original formulation for comparative test.

| | | |
|---|---|---|
| | Control | Experimental |
| Component | group | group |

| | | | | |
|---|---|---|---|---|
| Water-soluble | 6 | mM | 6 | mM |
| vitamin E | | | | |
| P-benzoquinone | 0.96 | mM | 0.96 | mM |
| Buffer system | Tris 100 | mM | Tris 100 | mM |
| Glucose | 100 | mM | 100 | mM |
| Glucose oxidase | 10 | U/mL | 10 | U/mL |

| | | |
|---|---|---|
| Acetonitrile | 30% | 30% |

| Triethylenediamine | 5 | mM | 0 | |
|---|---|---|---|---|
| (DABCO) | | | | |
| Sodium iodide | 5.25 | mM | 0 | |
| Ascorbic acid | 50 | mM | 50 | mM |
| Gallic acid | 20 | mM | 20 | mM |
| AMP | 5 | μM | 5 | μM |
| GMP | 5 | μM | 5 | μM |

| | | |
|---|---|---|
| Cyanuric acid | 0.0001% | 0.0001% |

The results are shown in the table below; there was no difference in the performance of each indicator before and after the removal of DABCO and NaI.

| | Signal | Signal | Imaging | Imaging | |
|---|---|---|---|---|---|
| Experimental | intensity_{—} | intensity_{—} | quality | quality | Error |
| conditions | Atto532 | Atto647N | score_Atto532 | score_Atto647N | rate |
| Control group | 172 | 346 | 2.95 | 2.15 | 6.50% |
| Experimental group | 177 | 341 | 3.07 | 2.11 | 6.39% |

### Example 34

Formulation 1:100 mM glucose, 10 U/mL glucose oxidase, 30 mM ascorbic acid, 1 mM gallic acid, water-soluble vitamin E (Trolox), 0.50 mM p-benzoquinone (BQ), 0.0001 wt % cyanuric acid, 5 μM 5′-adenosine monophosphate, 5 μM guanosine-5′-monophosphate, 200 mM tris(hydroxymethyl)aminomethane (Tris) and 30 v/v % acetonitrile, 15 v/v % liquid paraffin, pH 7.5.

Formulation 2:120 mM glucose, 12 U/mL glucose oxidase, 5 mM ascorbic acid, 15 mM ethyl gallate, 6 mM water-soluble vitamin E (Trolox), 0.0001 wt % cyanuric acid, 10 UM 5′-adenosine monophosphate, 10 μM guanosine-5′-monophosphate, 200 mM Tris, 20 mM NaCl and 20 v/v % acetonitrile, 10 v/v % liquid paraffin, pH 7.5. In preparing this reagent, water-soluble vitamin E was dissolved in acetonitrile, and other components were added after the solution was left to stand in the air for more than 10 h, thus completing the preparation.

Formulation 3:150 mM glucose, 15 U/mL glucose oxidase, 10 mM ascorbic acid, 10 mM propyl gallate, 5 mM hydroquinone, 12 mM water-soluble vitamin E (Trolox), 0.0005 wt % cyanuric acid, 2 μM 5′-adenosine monophosphate, 2 μM guanosine-5′-monophosphate, 250 mM Tris and 20 v/v % acetonitrile, 10 v/v % liquid paraffin, pH 8.0.

By referring to any one of the examples above, the three formulations were tested under the same other conditions. The results obtained from sequencing the same sample using the three formulations were all of good quality, as demonstrated by the close read length, throughput and error rate.

### Example 36

(Original) basic formulation disclosed in U.S. Pat. No. 7,282,337 or U.S. Pat. No. 7,666,593:134 μL of HEPES/NaCl, 24 μL of 100 mM water-soluble vitamin E Trolox (prepared with 2-(N-morpholine) ethanesulfonic acid, i.e., the MES buffer system, pH 6.1), 10 μL of triethylenediamine DABCO (prepared with MES, pH 6.1), 8 μL of 2 M glucose, 20 μL of NaI (50 mM, prepared with water), 4 μL of glucose oxidase and 30% acetonitrile.

New basic formulation: not containing 10 μL of triethylenediamine DABCO (prepared with MES, pH 6.1), optionally with or without NaI, with the rest the same as those in the basic formulation described above.

By referring to any one of the examples above, the two formulations were tested under the same other conditions. The results obtained from sequencing the same sample using both formulations were similar to those disclosed in that patent document, with both showing the capability of basically realizing single-molecule sequencing but with short read length and high error rate.

In the description of this specification, the description of the terms “one embodiment”, “some embodiments”, “schematic embodiments”, “examples”, “certain examples”, “specific examples”, or the like, means that the particular features, structures, materials, or characteristics described with reference to the embodiment or example are included in at least one embodiment or example of the present disclosure. In this specification, the schematic description of the aforementioned terms does not necessarily refer to the same embodiment or example. Moreover, the particular features, structures, materials, or characteristics described may be combined in any embodiment or example in any appropriate manner.

Although the embodiments of the present disclosure have been illustrated and described above, it will be appreciated that the aforementioned embodiments are exemplary and should not be construed as limiting the present disclosure, and that those of ordinary skills in the art can make changes, modifications, replacements, and variations to such embodiments, without departing from the scope of the present disclosure.

1-65. (canceled)

66. A method for incorporating a labeled nucleotide, comprising the following steps:
(a) providing a hybridization complex, wherein the hybridization complex is a hybrid of a primer and a template molecule, the primer being configured to hybridize to a 3′ end of the template molecule and the template molecule being a single-stranded nucleic acid molecule;
(b) subjecting a polymerase, a nucleotide analog and the hybridization complex to conditions suitable for a polymerization reaction to obtain an extension product by binding the nucleotide analog to the hybridization complex, the nucleotide analog comprising a sugar unit, a base, a cleavable blocking group and a fluorescent label linked together;
(c) replacing a solution system of (b) with a fluorescence detection reagent comprising an enzymatic oxygen scavenging system and a plurality of reducing agents and no triethylenediamine;
(d) irradiating at least a portion of the hybridization complex and acquiring at least a portion of a signal from the fluorescent label in the presence of the fluorescence detection reagent; and
(e) replacing the fluorescence detection reagent with a cleavage reagent to cleave the cleavable blocking group and the fluorescent label on the extension product, the cleavage reagent being used for cleaving the cleavable blocking group and the fluorescent label of the nucleotide analog.

67. The method according to claim 66, wherein the hybridization complex is attached to a substrate surface, and (d) comprises irradiating the substrate surface with light of a specific wavelength to excite fluorescence from the fluorescent label on the substrate surface and acquiring at least a portion of the fluorescence from the fluorescent label.

68. The method according to claim 67, wherein the fluorescent label comprises at least one in a combination of ROX, ATTO532 and Alexa fluor532 and at least one in a combination of CY5, IF700 and ATTO647N.

69. The method according to claim 68, wherein the enzymatic oxygen scavenging system is selected from a combination I comprising glucose and glucose oxidase, a combination II comprising glucose, glucose oxidase and catalase or a combination III comprising protocatechuic acid and protocatechuate 3,4-dioxygenase.

70. The method according to claim 69, wherein the reducing agent is selected from at least two of ascorbic acid, gallic acid, an analog or derivative of gallic acid, cyanuric acid and water-soluble vitamin E.

71. The method according to claim 70, wherein the fluorescence detection reagent comprises the combination I, ascorbic acid and gallic acid or the analog or derivative of gallic acid.

72. The method according to claim 71, wherein the fluorescence detection reagent comprises 50 mM-300 mM glucose, 2 U/mL-20 U/mL glucose oxidase, 1 mM-200 mM ascorbic acid and 1 mM-20 mM gallic acid or the analog or derivative of gallic acid.

73. The method according to claim 71, wherein the fluorescence detection reagent further comprises cyanuric acid.

74. The method according to claim 69, wherein the fluorescence detection reagent comprises the following components: 80 mM-150 mM glucose, 8 U/mL-12 U/mL glucose oxidase, 1 mM-200 mM ascorbic acid, 1 mM-15 mM gallic acid or ethyl gallate or propyl gallate, 6 mM-15 mM water-soluble vitamin E, 0.36 mM-0.96 mM p-benzoquinone, 0.0003 wt %-0.0009 wt % cyanuric acid, 4 μM-12 μM 5′-adenosine monophosphate, 4 μM-12 μM guanosine-5′-monophosphate, 100 mM-300 mM tris(hydroxymethyl)aminomethane and 18 v/v %-35 v/v % acetonitrile.

75. A fluorescence detection reagent, comprising an enzymatic oxygen scavenging system and a plurality of reducing agents and no triethylenediamine.

76. The reagent according to claim 75, wherein the enzymatic oxygen scavenging system is selected from a combination I comprising glucose and glucose oxidase, a combination II comprising glucose, glucose oxidase and catalase or a combination III comprising protocatechuic acid and protocatechuate 3,4-dioxygenase.

77. The reagent according to claim 76, wherein the plurality of reducing agents are selected from at least two of ascorbic acid, gallic acid, an analog or derivative of gallic acid, cyanuric acid and water-soluble vitamin E.

78. The reagent according to claim 77, wherein the fluorescence detection reagent comprises the combination I, ascorbic acid and gallic acid or the analog or derivative of gallic acid.

79. The reagent according to claim 78, wherein the fluorescence detection reagent comprises 50 mM-300 mM glucose, 2 U/mL-20 U/mL glucose oxidase, 1 mM-200 mM ascorbic acid and 1 mM-20 mM gallic acid or the analog or derivative of gallic acid.

80. The reagent according to claim 78, wherein the fluorescence detection reagent further comprises cyanuric acid.

81. The reagent according to claim 80, wherein the mass percent of the cyanuric acid is 0.0001%-0.001%.

82. The reagent according to claim 78, wherein the fluorescence detection reagent further comprises 1 μM-50 μM 5′-adenosine monophosphate and 1 μM-50 μM guanosine-5′-monophosphate.

83. The reagent according to claim 78, wherein the fluorescence detection reagent uses a Tris buffer system and has a pH of 6.5-8.5.

84. The reagent according to claim 76, wherein the fluorescence detection reagent comprises the following components: 80 mM-150 mM glucose, 8 U/mL-12 U/mL glucose oxidase, 1 mM-200 mM ascorbic acid, 1 mM-15 mM gallic acid or ethyl gallate or propyl gallate, 6 mM-15 mM water-soluble vitamin E, 0.36 mM-0.96 mM p-benzoquinone, 0.0003 wt %-0.0009 wt % cyanuric acid, 4 μM-12 μM 5′-adenosine monophosphate, 4 μM-12 μM guanosine-5′-monophosphate, 100 mM-300 mM tris(hydroxymethyl)aminomethane and 18 v/v %-35 v/v % acetonitrile.

85. A kit, comprising the fluorescence detection reagent and the nucleotide analog in the method according to claim 66.


SEQUENCE LISTING (4 sequences)

SEQ ID NO: 1 - DNA, 78 bp, synthetic construct
  misc_feature 1..73
  source 1..78: mol_type = other DNA
ctagactggacttgacgacttgcacgtagctagctgggtgcggaagagaaagaataccatgcagaaggaggcaaagta

SEQ ID NO: 2 - DNA, 77 bp, synthetic construct
  source 1..77: mol_type = other DNA
  misc_feature 1..77
gcagctaatgcatgcgcatctagagtcgtacgctgggtgcggaagagaaagaataccatgcagaaggaggcaaagta

SEQ ID NO: 3 - DNA, 78 bp, synthetic construct
  source 1..78: mol_type = other DNA
  misc_feature 1..73
tgacgtacctagactgagtcacgttcgacgatgctgggtgcggaagagaaagaataccatgcagaaggaggcaaagta

SEQ ID NO: 4 - DNA, 78 bp, synthetic construct
  source 1..78: mol_type = other DNA
  misc_feature 1..73
tcatctattatgccatctcccttgtggaatcagctgggtgcggaagagaaagaataccatgcagaaggaggcaaagta